(19) United States
(12) Patent Application Publication
(10) Pub. No.: US 2019/0060324 A1
(43) Pub. Date: Feb. 28, 2019

(54) POLYNUCLEOTIDE-POLY(DIOL) CONJUGATES, PROCESS OF PREPARATION AND USES THEREOF

(71) Applicant: THE ROYAL INSTITUTION FOR THE ADVANCEMENT OF LEARNING/MCGILL UNIVERSITY, Montreal (CA)

(72) Inventors: Hanadi F. SLEIMAN, Montreal (CA); Christopher J. SERPELL, Oxford (GB); Thomas G.W. EDWARDSON, Montreal (CA); Karina M.M. CARNEIRO, San Francisco, CA (US)

(21) Appl. No.: 16/182,956

(22) Filed: Nov. 7, 2018

Related U.S. Application Data
(63) Continuation of application No. 15/584,089, filed on May 2, 2017, now abandoned.
Application No. 15/584,089 is a continuation of application No. 14/623,911, filed on Feb. 17, 2015, now abandoned.
(60) Provisional application No. 61/940,657, filed on Feb. 17, 2014.

(51) Int. Cl. A61K 31/5377; A61K 47/54; C12N 15/11; C12N 15/113

(52) U.S. Cl. CPC A61K 31/5377; A61K 47/545; A61K 47/548; C12N 2320/32; C12N 2310/113; C12N 15/111; C12N 2310/351; C12N 2310/16; C12N 2310/141; C12N 2310/14; C12N 2310/11; C12N 15/113

(57) ABSTRACT

The present disclosure provides a conjugate of formula (I)

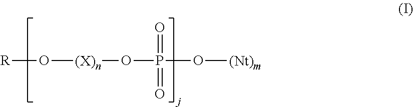

wherein each wherein each X, Nt, R, n, m and j are as defined herein. There is also provided a process for preparing a substantially monodisperse conjugate, a composition comprising said conjugate, and the use of a conjugate as defined herein in therapy.

### CROSS-REFERENCE TO RELATED APPLICATION(S) AND DOCUMENT(S)

This application is a continuation of U.S. patent application Ser. No. 15/584,089 filed on May 2, 2017 which is a continuation of U.S. patent application Ser. No. 14/623,911 filed on Feb. 17, 2015 which claims priority from U.S. provisional patent application Ser. No. 61/940,657 filed on Feb. 17, 2014. The content of the prior applications are herewith enclosed in their entirety.

### BACKGROUND OF THE DISCLOSURE

The key processes in molecular biology are executed by proteins and nucleic acids—natural sequenced polymers capable of storing data, and generating complex structure and function. Synthetic sequence-controlled polymers may find applications in the fields of data storage and biomedicine, and in the creation of materials with precisely tunable bulk properties and function (J.-F. Lutz, M. Ouchi, D. R. Liu, M. Sawamoto, Science 2013, 341).

One method to introduce molecular recognition into synthetic polymers is through biomolecule-polymer conjugation (M. Kwak, A. Herrmann, Chem. Soc. Rev. 2011, 40, 5745-5755), where a conventional synthetic polymer and an information-rich DNA or peptide/protein portion are covalently attached (J. Y. Shu, B. Panganiban, T. Xu, Annu. Rev. Phys. Chem. 2013, 64, 631-657). However, the synthesis of amphiphilic nucleic acid conjugates is fundamentally challenging, as it requires the end-to-end coupling of a highly charged DNA strand with a hydrophobic polymer chain.

### SUMMARY OF THE DISCLOSURE

In one aspect, there is provided a conjugate of formula (I)

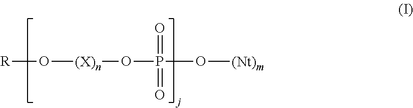

wherein each X is an alkylene chain of 1 to 20 carbon atoms independently selected n times, said alkylene chain carbon atoms being optionally interrupted with one or more of:
i) a cycloalkylene;
ii) an arylene or heteroarylene; and
iii) a heteroatom selected from O or N(R1) wherein R1 is H or a nitrogen substituent, provided that for each of said residue O—X—O, two or more consecutive N or O atoms are separated by two or more carbon atoms of said alkylene, cycloalkylene or arylene chain;
   each of said X is optionally independently substituted at any substitutable position by one or more substituent Rx; Nt is a nucleotide wherein one of a 3′ or 5′ sugar hydroxyl of the 3′ or 5′ end of said Nt is covalently bonded to the phosphorus (P) atom to form the phosphate linking group; R is H, a suitable hydroxyl protecting group; n is an integer; m is an integer; and j is an integer.

In some embodiments, the conjugates of formula I encompasses a di-, tri- or tetrablock copolymer which can be varied at will. Such block copolymer includes, but is not limited to, those having the following formula [O(X)ₙOPO₂]—[O(Y)ₙOPO₂]—[O(Z)nOPO₂], wherein X, Y and Z are the same or different block polymer.

In a further aspect, there is provided a process for preparing a substantially monodisperse conjugate as defined herein, comprising

1) contacting a compound having the general formula:

Rp-O—(X)ₙ—O—P(act)

wherein Rp is a suitable hydroxyl protecting group; P(Act) is a phosphorus (P)— based group suitable for forming a phosphate group when reacted with a hydroxyl (OH) group;
X and n are as defined herein; with a polynucleotide of formula (Nt)m, wherein one of a 3′ or 5′ end of said polynucleotide has one free hydroxyl (OH) group suitable to react with said P(Act) group; and
2) repeating step 1) above j−1 times; and
3) optionally deprotecting the group Rp.

In a further aspect, there is provided a composition comprising a conjugate as defined herein and acceptable additives.

In a further aspect, there is provided a use of a conjugate as defined herein in therapy.

In a further aspect, there is provided a method for encapsulating a molecule, comprising contacting said molecule with a conjugate as defined herein.

In a further aspect, there is provided a composition comprising a micelle and one or more compound; wherein said micelle is comprising a conjugate as defined herein, said conjugate having a hydrophilic segment formed by (Nt)ₘ as defined herein and a hydrophobic segment sufficient to form a micelle wherein the hydrophobic segment is comprising the residue of —O—(X)n-O— as defined herein, said hydrophobic segment forms a hydrophobic inner core and said hydrophilic segment forms the outer shell of said inner core; and wherein said compound is a hydrophobic compound encapsulated in said hydrophobic inner core.

In a further aspect, there is provided a method for treating a condition or illness in a subject in need thereof, comprising administering an effective amount of the composition as defined herein to a subject in need of treatment.

In a further aspect, there is provided a compound of formula (II)

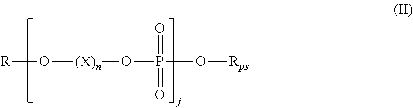

wherein each X is an alkylene chain of 1 to 20 carbon atoms independently selected n times, said alkylene chain carbon atoms being optionally interrupted with one or more of:
i) a cycloalkylene;
ii) an arylene or heteroarylene; and
iii) a heteroatom selected from O or N(R1) wherein R1 is H or a nitrogen substituent, provided that for each of said residue O—X—O, when two or more N or O atoms are present they are separated by two or more carbon atoms of said alkylene chain;
   each of said X is optionally independently substituted at any substitutable position by one or more substituent Rx;
   Rps is H, a suitable hydroxyl protecting group or is a solid support;
   R is H, a suitable hydroxyl protecting group;
   n is an integer; and
   j is an integer.

In a further aspect, there is provided a process for preparing a substantially monodisperse polymer of formula (II) comprising

1) contacting a compound having the general formula:

Rp-O—(X)ₙ—O—P(act)

wherein Rp is a suitable hydroxyl protecting group;
P(Act) is a phosphorus (P)— based group suitable for forming a phosphate group when reacted with a hydroxyl (OH) group or forming a covalent bond with a solid support;
X and n are as defined herein;
with a solid support (Rps) having groups suitable to react with said P(Act) group; and
2) repeating step 1) above j−1 times; and
3) optionally deprotecting the group Rp.

### BRIEF DESCRIPTION OF THE SEVERAL VIEWS OF THE DRAWINGS

FIG. 1 illustrates one general synthetic route for conjugates as defined herein using phosphoramidite chemistry on particular examples provided in this disclosure;

FIG. 2 is a reverse-phase HPLC trace of a two monomer conjugate obtained by synthesis without sequence control;

FIG. 3 is a reverse-phase HPLC traces of purified HEₓ-DNA conjugates;

FIG. 4 are reverse-phase HPLC traces of sequence defined with respect to the DNA portion;

FIG. 5 And FIG. 6 are fluorescence spectrums of Nile Red in the presence of conjugates as defined herein.

FIG. 7 represents gel electrophoresis of RNA conjugates;

FIG. 8 are representative HPLC traces of HE₁₂-RNA versus unmodified control;

FIG. 9 is a schematic representation of the encapsulation of a hydrophobic compound in micelles as defined herein and the chemical formula of BKM120 and NU7026;

FIG. 10 (a)-(c) are HPLC traces of purified encapsulation products in HE₁₂-RNA micelles;

FIG. 11 is an absorbance spectra for BKM120 encapsulation;

FIG. 12 Illustrates the release kinetics of BKM120 from BKM120-loaded HE₁₂-DNA micelles;

FIG. 13 Characterize the In vitro micelle stability studies in physiologically relevant conditions;

FIG. 14 MTS assay to measure the cytotoxity of BKM 120-loaded HE₁₂-DNA micelles;

FIG. 15 Annexin V/PI apoptosis assay on chronic lymphocytic leukemia (CLL) primary patient cells

FIG. 16 are graphs representing cleaved caspase-3 apoptosis assay on chronic lymphocytic leukemia (CLL) primary patient cells;

FIG. 17 (a)-(d) are HPLC traces of crude product mixtures of terminal modification;

FIG. 18 (a)-(c) are HPLC traces of crude product mixtures of internal modification;

FIG. 19 is the UV absorbance study of sequence-controlled polymers; and

FIG. 20 (a)-(d) are ESI/IT-MS data for sequence-controlled polymers.

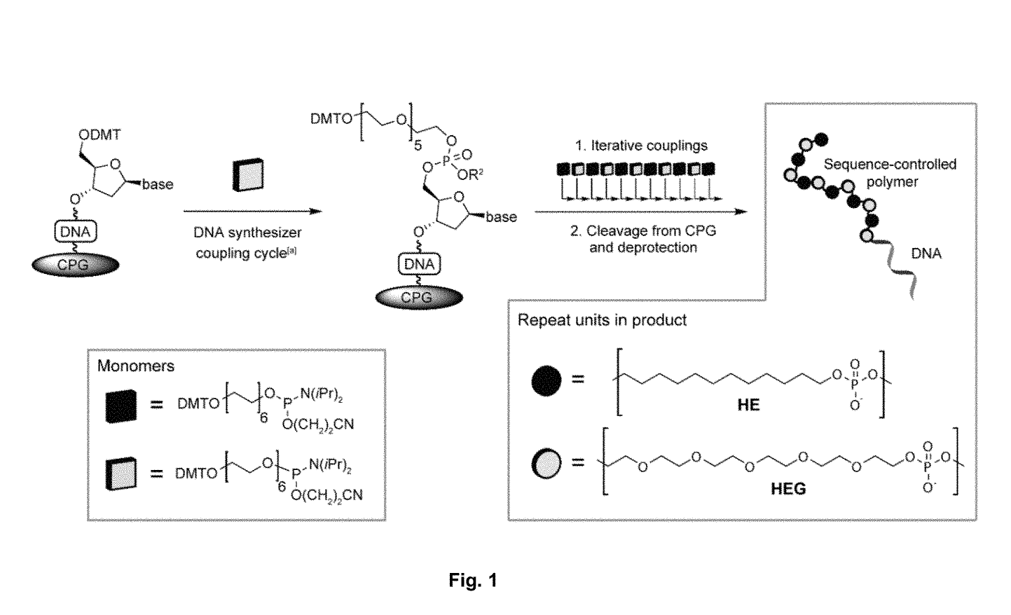

### DETAILED DESCRIPTION OF THE DISCLOSURE

DNA amphiphiles in particular are especially attractive, because they can self-assemble into a variety of morphologies through microphase separation while retaining the ‘smart’ and addressable biomolecule component (see a) M.-P. Chien, A. M. Rush, M. P. Thompson, N. C. Gianneschi, Angew. Chem. Int. Ed. 2010, 49, 5076-5080; b) O. Pokholenko, A. Gissot, B. Vialet, K. Bathany, A. Thiery, P. Barthelemy, J. Mater. Chem. B 2013, 1, 5329-5334; c) L. Wang, Y. Feng, Z. Yang, Y.-M. He, Q.-H. Fan, D. Liu, Chem. Commun. 2012, 48, 3715-3717; d) K. M. M. Carneiro, G. D. Hamblin, K. D. Hanni, J. Fakhoury, M. K. Nayak, G. Rizis, C. K. McLaughlin, H. S. Bazzi, H. F. Sleiman, Chem. Sci. 2012, 3, 1980-1986; e) T. G. W. Edwardson, K. M. M. Carneiro, C. K. McLaughlin, C. J. Serpell, H. F. Sleiman, Nature Chem. 2013, 5, 868-875) and can result in attractive platforms for functional nucleic acids such as aptamers and silencing RNA (see a) Y. Wu, K. Sefah, H. Liu, R. Wang, W. Tan, Proc. Natl. Acad. Sci. U.S.A. 2010, 107, 5-10; b) M. Raouane, D. Desmaële, G. Urbinati, L. Massaad-Massade, P. Couvreur, Bioconjugate Chem. 2012), and in membrane anchoring and functionalization tools (see a) J. G. Woller, J. K. Hannestad, B. Albinsson, J. Am. Chem. Soc. 2013, 135, 2759-2768; b) E. P. Lundberg, B. Feng, A. Saeid Mohammadi, L. M. Wilhelmsson, B. Norden, Langmuir 2013, 29, 285-293; c) Y.-H. Chan, B. Lengerich, S. Boxer, Biointerphases 2008, 3, FA17-FA21).

The present disclosure provides a process for the production of novel conjugates, in particular monodisperse conjugates. This is achieved by attaching well-defined blocks one at a time to a nucleotide (e.g., DNA-based, RNA-based or DNA/RNA-based), and thus allows the control over the length of the modification. The process can be used to create synthetic sequence-defined polymers, offering flexibility to vary the self-assembly and encapsulation properties by variation of the sequence of monomers.

Control of sequence-specific hydrophobic intra- or intermolecular aggregation within a polymer is an aspect and the present disclosure allows for obtaining synthetic mimics of the complex structures and functions exhibited by biological sequenced polymers.

As used herein, polynucleotide moiety of the conjugate contemplated are not especially limited. The nucleotide or polynucleotide should preferably have a useful function such as any of those described herein. The nucleotide can be a natural or non-natural (synthetic) nucleotide composed of a natural or non-natural nucleobase (such as purine or pyrimidine bases), a natural or non-natural (including five-carbon sugars such as ribose or 2-deoxyribose, six- or larger carbon sugar rings), and one or more phosphate groups linking consecutive nucleotides. The phosphate groups may either be at the 2, 3, or 5-carbon position of the sugar, preferably the 5-carbon position. Exemplary natural nucleotides includes, but are not limited to, adenine, guanine, cytosine, thymine and uracil. Exemplary non-modified nucleotides include, but are not limited to, 4-acetylcytidine, 5-(carboxyhydroxymethyl) uridine, 2′-O-methylcytidine, 5-carboxymethylaminomethyl-2-thiouridine, 5-carboxymethylaminomethyluridine, dihydrouridine, 2′-O-methylpseudouridine, D-galactosylqueuosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, D-mannosylqueuosine, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, pseudouridine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine and 3-(3-amino-3-carboxy-propyl)uridine.

The bases and sugars should be compatible with nucleotide coupling chemistry (such as phosphoramidite chemistry). The nucleotide segment(s) could be any length from 1 nucleotide to over 100, preferably more than 2, preferably more than 3, preferably more than 5 or preferably more than 10. In some complementary embodiments, the nucleotide segment(s) could be any length shorter than 50 nucleotides, 40 nucleotides, 30 nucleotides, 25 nucleotides or 20 nucleotides. The limiting factor will only be the yield achievable as it is a function of the length of the polynucleotide. The preferred ranges will be those which are high yielding and will depend on the application (for example siRNA or antisense short 19-22 mers may be needed whereas longer strands may be used to produce more stable duplex forming structures). For applications where amphiphilic assembly is desired (micelles/vesicles etc.) it is likely that shorter DNA strands will also be more useful, as the ratio of hydrophobic to hydrophilic block are important determinants of aggregation.

In some embodiments, the nucleotide can include various modifications for example in the phosphodiester linkage and/or on the sugar, and/or on the base. For example, the oligonucleotide can include one or more phosphorothioate linkages, phosphorodithioate linkages, and/or alkyl or arylphosphonate linkages. Additional useful modifications include, without restriction, modifications at the 2′-position of the sugar, such as 2′-O-alkyl modifications such as 2′-O-methyl modifications, 2′-amino modifications, 2′-halo modifications such as 2′-fluoro; acyclic nucleotide analogs. In another embodiment, the oligonucleotide has modified linkages throughout, e.g., phosphorothioate; has a 3′- and/or 5′-cap; includes a terminal 3′-5′ linkage; the oligonucleotide is or includes a concatemer consisting of two or more oligonucleotide sequences joined by a linker(s).

Such modified nucleotide segment backbones include, for example, phosphorothioates, chiral phosphorothioates, phosphorodithioates, phosphotriesters aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, carboranyl phosphate and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity typically include a single 3′ to 3′ linkage at the 3′-most internucleotide linkage i.e. a single inverted nucleoside residue which may be abasic (the nucleobase is missing or has a hydroxyl group in place thereof).

Some exemplary modified nucleotide backbones that do not include a phosphodiester linkage have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Modified nucleotide segment may also contain one or more substituted sugar moieties. For example, such oligonucleotides can include one of the following 2′-modifications: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl, or 2′-O—(O-carboran-1-yl)methyl. Particular examples are O[(CH₂)ₙO]ₘCH₃, O(CH₂)˜OCH₃, O(CH₂)ₙNH₂, O(CH₂)ₙCH₃, O(CH₂)ₙONH₂, and O(CH₂)ₙON [(CH₂)ₙCH₃)]₂, where n and m are from 1 to 10. Other exemplary modified sugar moities include one of the following 2′-modifications: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃. OCF₃, SOCH₃, SO₂CH₃, ONO₂, NO₂, N₃, NH₂, heterocycloalkyl, heterocycloalkaryl, aminoalkylamino, polyalkylamino, substituted silyl, a reporter group, an intercalator, a group for improving the pharmacokinetic properties of an ON, or a group for improving the pharmacodynamic properties of an ON. Examples include 2′-methoxyethoxy (2′-O—CH₂CH₂OCH₃, also known as 2′-O-(2-methoxyethyl) or 2′-MOE, an alkoxyalkoxy group; 2′-dimethy-laminooxyethoxy, i.e., a O(CH₂)₂ON(CH₃)₂ group, also known as 2′-DMAOE; and 2′-dimethylaminoethoxyethoxy (also known as 2′-O-dimethylaminoethoxyethyl or 2′-DMAEOE), i.e., 2′-O—CH₂—O—CH₂—N(CH₂)₂.

Other modifications include Locked Nucleic Acids (LNAs) in which the 2′-hydroxyl group is linked to the 3′ or 4′ carbon atom of the sugar ring thereby forming a bicyclic sugar moiety. The linkage can be a methylene (—CH₂—)˜ group bridging the 2′ oxygen atom and the 4′ carbon atom wherein n is 1 or 2. Further LNAs include sulfur, oxygen or nitrogen bridge modifications, (Oxy-LNA, amino-LNA and thio-LNA). Further modifications include 2′-methoxy (2′-O—CH₃), 2′-methoxyethyl (2′O—CH₂—CH₃), 2′-ethyl, 2′-ethoxy, 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F).

The 2′-modification may be in the arabino (up) position or ribo (down) position. Similar modifications may also be made at other positions on the nucleotide segment, particularly the 3′ position of the sugar on the 3′ terminal nucleotide or in 2′-5′ linked oligonucleotides and the 5′ position of the 5′ terminal nucleotide. Nucleotide segments may also have sugar mimetics such as cyclobutyl moieties in place of the pentofuranosyl sugar.

The preparation of protected/activated residues of formula Rp-O—CH₂—(X)n-CH₂—O—P(act) or Rp-O—(X)—O—P(act) as described herein is well documented and has been part of the general knowledge of oligonucleotide synthesis (see Niu, J.; Hili, R.; Liu, D. R. Nat Chem 2013, 5, 282-292). The diols are normally preferably free of chemical groups that may be nucleophile (e.g. amine, alcohol . . . ) unless those can be suitably protected and resistant to the coupling conditions. As such the diols can contain any functionality compatible with the standard oligonucleotide protocols for synthesis, cleavage, and deprotection. The functionalities must therefore generally not be vulnerable to degradation by trichloroacetic acid, iodine, or ammonium hydroxide. They should also not leave free alcohols or amines separate from the main chain from which branching could occur—such moieties should be protected using base-cleavable groups. In particular, alkyne (for use in CuAAC click chemistry), alkene (for cross-linking through polymerisation or thiol-ene reactions), amide (as a robust linkage), and dithio (precursors to thiols for cross-linking through sulphur bridges) groups are well tolerated.

In one embodiment, a conjugate of this description has the formula (Ia)

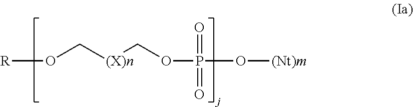

wherein each X, Nt, R, n, m and j are as defined herein and provided that for each of said residue O—CH₂—X—CH₂—O, two or more consecutive N or O atoms are separated by two or more carbon atoms of said alkylene, cycloalkylene or arylene chain. Preferably, X is one or more of Xa-Xi.

In general, the diol is first treated with dimethoxytrityl chloride in dry pyridine to give a mix of mono, bis, and un-protected products, from which the mono-protected compound is isolated. This is then reacted with 2-cyanoethyl N,N-diisopropylchlorophosphoramidite in the presence of DMAP and DIPEA, in a non-protic solvent. Reaction success is usually assessed by the presence of a ³¹P NMR peak at 148 ppm. Purification of the protected/activated diol can be achieved through column chromatography, in particular on basic alumina or base-treated silica, though it is often possible to react directly with DNA on solid support.

Examples of X include, without limitation:

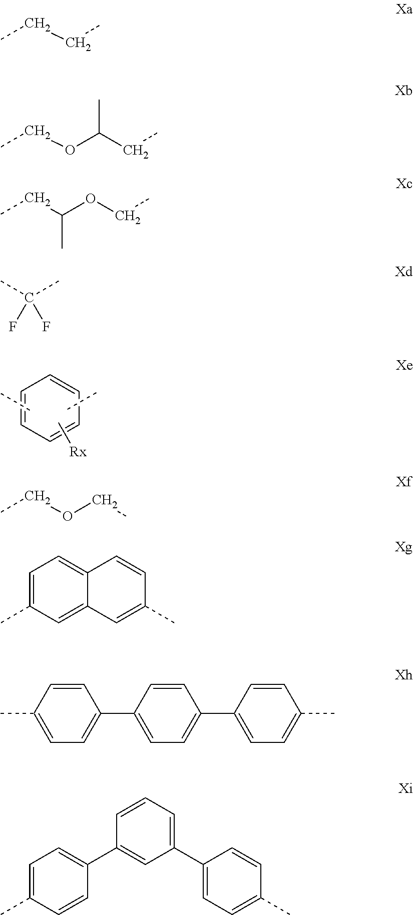

In one embodiment, the alkylene chain of X or i) the cycloalkylene or ii) arylene are each optionally substituted by one or more of halo (preferably a fluoro atom), alkyl of 1 to 6 carbons, aryl of 6 to 10 ring members, —COOH or —C(O)NH-alkyl, protected amino, protected hydroxyl, alkene or a dithio group (—S—S—RI).

Preferably, the process of the present disclosure is carried on solid supports. There are various solid support available and the DNA solid support resins, designed to be used for phosphoramidite chemistry, are especially preferred in practice of the present disclosure. This includes universal, nucleoside derivatised (A,C,G,T) and specialist (amino, fluorophore etc.) supports. Additionally most of these solid supports are available in different pore sizes: 500 Å suitable for up to 40 bases, 1000 Å suitable for up to 100 bases and 2000 Å for longer strands.

Therefore correct choice of pore size can be used to improve synthetic yields of DNA-polymer conjugates. Additionally the loading of reactive sites per gram of solid support can also be modulated to enable larger scale syntheses or to achieve higher yields (longer strands will need lower loading as steric hindrance becomes an issue with high-loading and long strand synthesis).

Examples of solid support include universal Controlled-pore glass (CPG) solid supports for DNA synthesis (many being available commercially): (1) N-Methyl-succinimido[3,4-b]-7-oxabicyclo[2.2.1]heptane-6-(4,4′-dimethoxytrityloxy)-5-succinoyl long chain alkylamino CPG, (2) Unylinker™ CPG (commercially available from Chemgenes corporation), (3) N-Methyl-succinimido[3,4-b]-7-oxabicyclo[2.2.1]heptane-6-(4,4′-dimethoxytrityloxy)-5-succinoyl long chain alkylamino CPG, (4)1-Dimethoxytrityloxy-2-O-dichloroacetyl-propyl-3-N-succinyl-CPG

Examples of Universal Highly-crosslinked polystyrene (PS) bead supports for DNA synthesis include (many being available commercially): (1) N-Methyl-succinimido[3,4-b]-7-oxabicyclo[2.2.1]heptane-6-(4,4′-dimethoxytrityloxy)-5-succinoyl-polystyrene, (2) 1-Dimethoxytrityloxy-2-O-dichloroacetyl-propyl-3-N-ureayl-polystyrene, (3) Unylinker™ Polystyrene (commercially available from Chemgenes corporation)

Examples of functionalized supports for DNA synthesis (many being available commercially) include: (1) 3-Dimethoxytrityloxy-2-(3-(6-carboxy-(di-O-pivaloyl-fluorescein)propanamido)propyl)-1-O-succinyl-long chain alkylamino-CPG, (2) 1-O-Dimethoxytrityl-3-oxahexyl-disulfide, 1′-succinoyl-long chain alkylamino-CPG, (3) 3-Dimethoxytrityloxy-2-(3-(fluorenylmethoxycarbonylamino)propanamido)propyl-1-O-succinyl-long chain alkylamino-CPG, (4) 1,2-Dithiane-4-O-dimethoxytrityl-5-succinoyl-long chain alkylamino-CPG, (5) 3-Dimethoxytrityloxy-2-(3-((4-t-butylbenzoyl)-biotinyl)propanamido) propyl-1-O-succinyl-long chain alkylamino-CPG, (6) 3-Dimethoxytrityloxy-2-(3-(3-propargyloxypropanamido)propanamido)propyl-1-O-succinoyl long chain alkylamino CPG

In carrying out the process of the present disclosure, protecting groups may be typically used either on the nucleotide or diol. Different protecting groups could also be employed such as Fmoc, Trityl and monomethoxy trityl (MMT). Trityl and MMT are commonly introduced as a more stable substitution of dimethoxy trityl (DMT) for reagents where stability is a concern, removed with trichloroacetic acid rather than dichloroacetic acid.

Fmoc can be introduced as an orthogonal protecting group, removed with 1,8-Diazabicycloundec-7-ene. This can be used to introduce branch points in the growing chain structure.

In carrying out the process of the present disclosure, coupling conditions/reagents will be used to link consecutive nucleotides or diols. The use of phosphoramidite coupling is suitable to produce phosphate punctuated polymers. Other coupling reactions may be used.

In certain embodiments, m is an integer of from 10 to 25.

In certain embodiments, n is an integer of from 3 to 20 or n is an integer of from 5 to 10 or n is an integer of 4 to 6 or n is an integer of 3 to 5.

In certain embodiments J is an integer of from 3 to 20 or J is an integer of from preferably 5 to 15.

In one embodiment, m is an integer of from 10 to 25; n is an integer of from 3 to 20, preferably 5 to 10; J is an integer of from 3 to 20, preferably 5 to 15.

In one embodiment, m is an integer of from 10 to 25; n is an integer of 4 to 6; J is an integer of from 5 to 15.

In one embodiment, m is an integer of from 10 to 25; n is an integer of 3 to 5; J is an integer of from 5 to 15.

In one embodiment, n is an integer of from 3 to 20, preferably 5 to 10; J is an integer of from 3 to 20, preferably 5 to 15.

In one embodiment, n is an integer of 4 to 6; J is an integer of from 5 to 15.

In one embodiment, n is an integer of 3 to 5; J is an integer of from 5 to 15.

In one embodiment, in a conjugate as defined herein, R is H or a suitable hydroxyl protecting group; X is —CH2-CH2- or —CH2-O-CH2-; Nt is comprising C, G, A, T, and/or U; m is an integer of from 10 to 25; n is an integer of from 3 to 20, preferably 5 to 10; J is an integer of from 3 to 20, preferably 5 to 15.

In one embodiment, in a conjugate as defined herein, R is H or a suitable hydroxyl protecting group; X is —CH2-CH2-; Nt is C, G, A, T, and/or U; m is an integer of from 10 to 25; n is an integer of 4 to 6; J is an integer of from 5 to 15.

In one embodiment, in a conjugate as defined herein, R is H or a suitable hydroxyl protecting group; X is —CH2-O-CH2-; Nt is comprising C, G, A, T, and/or U; m is an integer of from 10 to 25; n is an integer of 3 to 5; J is an integer of from 5 to 15.

In one embodiment, in a conjugate as defined herein, R is H, or a protecting group Fmoc, Trityl, monomethoxy trityl (MMT) or dimethoxy trityl (DMT).

In one embodiment, in a conjugate as defined herein X is an alkylene chain substituted by Rx which is a F atom.

In one embodiment, in a conjugate as defined herein X is an alkylene chain interrupted by —O-(arylene or heteroarylene)-O—.

In one embodiment, in a conjugate as defined herein X is dodecane, hexaethyloxy,

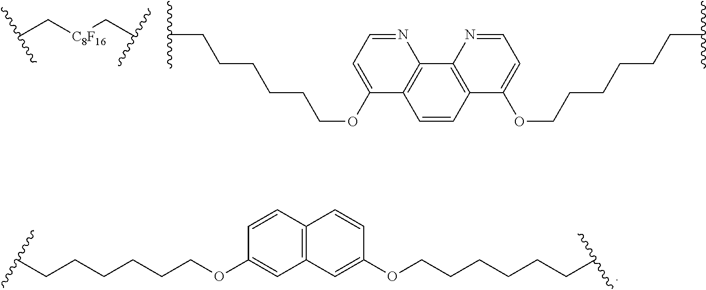

In one embodiment, in a conjugate as defined herein (Nt)ₘ is a therapeutic nucleotide segment.

In one embodiment, in a conjugate as defined herein (Nt)ₘ is comprising a silencing mRNA (siRNA), antisense oligonucleotides, micro RNA (miRNA), antagomir (anti-miRNA), aptamer or concatomer.

The term “alkyl”, as used herein, is understood as referring to a saturated, monovalent unbranched or branched chain of 1-10 carbon atoms, alternatively 1-6 or 1-3 carbon atoms.

The term “alkylene” means a bivalent radical derived from an alkyl.

The term “aryl”, as used herein, is understood as referring to 5-, 6- and 7- or more membered aromatic groups, for example phenyl or naphthyl. The term “arylene” means a bivalent radical derived from an aryl.

The term “cycloalkyl” refers to a saturated cyclic moiety having three or more carbon atoms; preferably from three to six carbon atoms. The term “cycloalkylene” means a bivalent radical derived from a cycloalkyl.

The term “substituent Rx” is comprising for example halogen (in particular F), alkyl, cycloalkyl, amino (—NH2) or preferably protected amino, amido (such as —NH(CO)-alkene or —NH(CO)— alkylthiol), hydroxyl or preferably protected hydroxyl.

The term “heteroaryl” or “heteroarylene” represents a 3 to 11 membered optionally substituted aromatic cyclic moiety wherein said cyclic moiety is interrupted by at least one heteroatom selected from oxygen (O), sulfur (S) or nitrogen (N). Heteroaryls may be monocyclic or polycyclic rings. Heteroaryls may be 3 to 6 membered monocyclic ring or 5 to 6 membered monocyclic ring. Heteroaryls may be 7 to 12 membered bicyclic ring or 9 to 10 membered bicyclic ring

The substituent R1 (or nitrogen substituent) is comprising for example alkyl, cycloalkyl, CH2(CO)NH-alkene or —CH2(CO)NH-alkylthiol.

Nitrogen or hydroxyl protecting groups are well known in the art. Suitably, the protecting groups should be compatible with the oligonucleotide protocols for synthesis, cleavage, and deprotection.

The conjugates described herein can be used for the encapsulation and/or delivery of various cargo. As indicated above, in some embodiments, the conjugate can be designed to further bear a peptide that can be used to target the conjugate to a specific cell, tissue or organ, to facilitate the entry of the conjugate into a specific cell (or into the nucleus) or transfer across a biological membrane (e.g., the blood-brain barrier for example), and/or to induce self-assembly of nanoparticles that have improved pharmacokinetics.

In one embodiment, the conjugates can be used to deliver the nucleotide segment to a cell (e.g., and in some further embodiment, inside a cell). For example, a plurality of (preferably monodispersed) conjugates can be admixed to form of particles having a core (e.g., mainly consisting of the repeating unit —[O—(X)ₙ—O—P(O)₂-]ⱼ- or —[O—CH₂—(X)ₙ—O—CH₂—P(O)₂—]ⱼ— of conjugates) from which the nucleotide segments extend outwardly. Particles formed with the various combination of conjugates can be used to delivery nucleotide segments having themselves therapeutic uses, such as, for example, silencing mRNA (siRNA), antisense oligonucleotides, micro RNA (miRNA), antagomir (anti-miRNA), aptamer, concatomer, peptide mimics, etc. In some embodiments, the particles described herein can be used to deliver nucleotide segment(s) in the absence of other carrier entities (such as liposomes, polycationic amines, dendrimers, and lipid-based agents (e.g., cationic lipids) for example). In other embodiments, the particles described herein can be obtained without the need of chemically cross-linking the various conjugate entities together to provide the delivery of the nucleotide segment.

In another embodiment, the conjugates can be used to encapsulate (and ultimately deliver) therapeutic agents (for therapeutic applications) or diagnostic agents (for diagnostic/imaging applications for example). In such embodiment, a plurality of conjugates are admixed with one or a combination of therapeutic/diagnostic agents to form particles encapsulating the therapeutic/diagnostic agent. In one specific embodiment, the repeating unit —[O—(X)ₙ—O—P(O)₂-]ⱼ— or —[O—CH₂—(X)ₙ—O—CH₂—P(O)₂—]ⱼ— of the conjugates form a surrounding core around the therapeutic/diagnostic agent and the nucleotide segment extends outwardly from this surrounding core. This may be useful for the encapsulation of lipophilic therapeutic/diagnostic agents. In some embodiments, the particles containing the lipophilic/diagnostic agent can be formed in the absence of other carrier entities. In other embodiments, the particles containing the lipophilic/diagnostic agent described herein can be obtained without the need of chemically cross-linking distinct conjugates. In other embodiments, the particles containing the hydrophilic/diagnostic agent described herein can be obtained without the need of chemically cross-linking distinct conjugates.

The therapeutic agents or diagnostic agents encapsulated in a micelle formed by the conjugates described herein can be useful for the delivery of such agents to any animal, but preferably mammals, and most preferably humans.

Preferably, the therapeutic agents or diagnostic agents is a hydrophobic agent. Hydrophobic agents are preferably those with high octanol/water partition coefficients (i.e. preferentially distributed to hydrophobic solvent octanol). The hydrophobic agent include anticancer agents, antifungal agents, antibiotics such as a macrolide, steroids, anti-inflammatory agents, antiviral agents and vitamins. The hydrophobic drug can be selected from the group consisting of a taxane analog, an anthracycline analog (such as doxorubicin), camptothecin, 5-fluorouracil, BKM120 and NU7026.

Preferably, the conjugates useful for encapsulating a hydrophobic agent in a micelle as described herein have a hydrophilic segment and a hydrophobic segment. The micelle has the hydrophilic segment as its outer shell and in its hydrophobic inner core a hydrophobic agent such as a hydrophobic drug. In one embodiment, said conjugate is comprising a hydrophilic segment comprising (Nt)m and a hydrophobic segment sufficient to form a micelle to encapsulate said compound, wherein the hydrophobic segment is comprising the residue of —O—(X)n-O—. In one embodiment, in said conjugate, R is H, (X)n is (—CH2CH2-)6, j is an integer of 6 or more and m is an integer of from 10 to 25.

In one embodiment of the method for treating a condition or illness in a subject in need thereof, said condition or illness is cancer.

In another embodiment, the conjugates can be designed with specific chain structures which present moieties capable of allowing specific intramolecular folding. Thus giving way to rationally designed architectures which may act as protein mimics. This may be useful for studying the dynamics of the folding induced by non-covalent interactions.

In another embodiment, the conjugates may be used to create delivery devices which are stimuli responsive, by introduction of functional groups which will affect self-assembly based on an environmental change such as pH or redox potential.

### General

Magnesium acetate, acetic acid, tris(hydroxymethyl)-aminomethane (Tris), formamide, urea, Nile Red and were used as purchased from Sigma-Aldrich. Acetic acid and boric acid were purchased from Fisher Scientific and used without further purification. GelRed™ nucleic acid stain was purchased from Biotium Inc. Acetone ACS reagent grade was purchased from Fisher. Ammonium citrate dibasic and 3-hydroxypicolinic acid were purchased from Aldrich. Acrylamide/Bis-acrylamide (40% 19:1 solution) and TEMED were obtained from Bioshop Canada Inc. and used as supplied. 1 μmol Universal 1000 Å LCAA-CPG supports and standard reagents used for automated DNA synthesis were purchased through Bioautomation. Sephadex G-25 (super fine, DNA grade) was purchased from Glen Research. TAMg buffer is composed of 45 mM Tris and 12.5 mM Mg(OAc)₂.6H₂O with pH adjusted to 8.0 using glacial acetic acid.

### Instrumentation

Standard automated oligonucleotide solid-phase synthesis was performed on a Mermade MM6 synthesizer from Bioautomation. HPLC purification was carried out on an Agilent Infinity 1260. DNA quantification measurements were performed by UV absorbance with a NanoDrop Lite spectrophotometer from Thermo Scientific. A Varian Cary 300 Bio spectrophotometer was used for melting temperature studies. Gel electrophoresis experiments were carried out on a 20×20 cm vertical Hoefer 600 electrophoresis unit. Gel images was captured using a ChemiDoc™ MP System from Bio-Rad Laboratories. Thermal annealing of all DNA structures was conducted using an Eppendorf Mastercycler® 96 well thermocycler. Mass determination was carried out using Matrix-Assisted Laser Desorption/Ionization Time-Of-Flight Mass Spectrometry (MALDI-TOF-MS) on a Bruker Autoflex™ III MALDI-TOF mass spectrometer. Liquid Chromatography Electrospray Ionization Mass Spectrometry (LC-ESI-MS) was carried out using Dionex Ultimate 3000 coupled to a Bruker MaXis Impact™ QTOF. Fluorescence emission spectra were obtained using an Agilent Cary Eclipse Fluorescence Spectrophotometer.

### Synthesis, Purification and Characterization of Sequence-Defined Conjugates

In the following example, an oligonucleotide of mixed sequence, comprising of 19 nucleotides, was first synthesized on the solid support using standard automated procedures. Next, certain diols were appended to the oligonucleotide chain, using the same automated phosphoramidite chemistry.

Commercially available dimethoxytrityl (DMT) protected dodecane-diol phosphoramidite could be used in the following particular examples.

The diol which corresponds to a hexameric section of poly(ethylene)—here labelled HE, hexaethylene. The resulting conjugate therefore consists of a DNA portion functionalised at its 5′ terminus with a series of HE units punctuated by phosphates. Several conjugates were prepared by modifications to the 5′ terminus of the DNA strand with 1 to 12 successive additions of HE units. It was found that 12 units were attached to the oligonucleotide portion with good efficiency, by monitoring the DMT absorbance after each coupling cycle.

Applicant has also shown that the present process is flexible enough to allow for using different diols either in sequence or block. In the following examples, a mixed system of hydrophobic HE blocks and hydrophilic hexaethylene glycol (HEG, also available as a commercial DMT-protected phosphoramidite reagent) blocks could be synthesized and the properties of the resultant sequence polymers assessed. Five different modified oligonucleotides, each containing a 5′ modification of 12 units in length (six HE, six HEG) were successfully synthesized. Each of these DNA strands differs in the pattern of the HE and HEG units, being grouped into blocks of one, two, three, and six oligomers. For the blocks of six, the position with respect to the DNA was also varied, producing two triblock architectures, HEG₆-HE₆-DNA and HE₆-HEG₆-DNA.

### Solid-Phase Synthesis of HE/HEG-DNA Conjugates

DNA synthesis was performed on a 1 μmole scale, starting from a universal 1000 Å LCAA-CPG solid-support. Coupling efficiency was monitored after removal of the dimethoxytrityl (DMT) 5′-OH protecting groups. DMT-dodecane-diol (cat.# CLP-1114) and DMT-hexaethyloxy Glycol (cat.# CLP-9765) phosphoramidites were purchased from Chemgenes. DMT-hexaethyloxy Glycol and DMT-dodecane-diol amidites were dissolved in the appropriate solvent under a nitrogen atmosphere in a glove box (<0.04 ppm oxygen and <0.5 ppm trace moisture). For DMT-hexaethyloxy Glycol (0.1M, anhydrous acetonitrile) and DMT-dodecane-diol (0.1M, anhydrous dichloromethane) amidites extended coupling times of 5 minutes were used respectively using 0.25M 5-(Ethylthio)tetrazole in anhydrous acetonitrile. Removal of the DMT protecting group was carried out using 3% dichloroacetic acid in dichloromethane by DNA on the DNA synthesizer. Completed syntheses were cleaved from the solid support and deprotected in 28% aqueous ammonium hydroxide solution for 16-18 hours at room temperature. The crude product solution was separated from the solid support and concentrated under reduced pressure at 60° C. This crude solid was re-suspended in 1 mL Millipore water. Sephadex G-25 column and 0.22 μm centrifugal filter were then performed prior to HPLC purification. The resulting solution was quantified by absorbance at 260 nm.

TABLE 1
Sequences used for conjugates.

| | | SEQ |
|---|---|---|
| Molecule | Sequence (5′-xx-3′) | ID NO: |
| DNA | TTTTTCAGTTGACCATATA | 1 |
| HE₁-DNA | DTTTTTCAGTTGACCATATA | 1 |
| HE₂-DNA | DDTTTTTCAGTTGACCATATA | 1 |
| HE₃-DNA | DDDTTTTTCAGTTGACCATATA | 1 |
| HE₄-DNA | DDDDTTTTTCAGTTGACCATATA | 1 |
| HE₅-DNA | DDDDDTTTTTCAGTTGACCATATA | 1 |
| HE₆-DNA | DDDDDDTTTTTCAGTTGACCATATA | 1 |
| HE₁₂-DNA | DDDDDDDDDDDDTTTTTCAGTTGAC | 1 |
| | CATATA | |
| (HE-HEG)₆-DNA | DHDHDHDHDHDHTTTTTCAGTTGAC | 1 |
| | CATATA | |
| (HE₂-HEG₂)₃-DNA | DDHHDDHHDDHHTTTTTCAGTTGAC | 1 |
| | CATATA | |
| (HE₃-HEG₃)₂-DNA | DDDHHHDDDHHHTTTTTCAGTTGAC | 1 |
| | CATATA | |
| HE₆-HEG₆-DNA | DDDDDDHHHHHHTTTTTCAGTTGAC | 1 |
| | CATATA | |
| HEG₆-HE₆-DNA | HHHHHHDDDDDDTTTTTCAGTTGAC | 1 |
| | CATATA | |
| Acomp | TATATGGTCAACTG | 2 |

(D = residue obtained from reaction of DMT-dodecane-
diol amidite),
(H = residue obtained from reaction of DMT-
hexaethyloxy Glycol amidite).

Through standard coupling (e.g. phosphoramidite coupling), it was possible to produce nucleic acid conjugate mimics in excellent yields with total control over the degree of polymerization (up to 72 units), leading to molecularly monodisperse products. As described below, the presence of phosphate moieties in the polymer backbone does not hinder the hydrophobic properties of the polymer section, producing spherical micelles capable of encapsulating guests.

### HPLC Purification

Solvents (0.22 μm filtered): 50 mM Triethylammonium acetate (TEAA) buffer (pH 8.0) and HPLC grade acetonitrile. Elution gradient: 3-70% acetonitrile over 30 minutes at 60° C. Column: Hamilton PRP-C18 5 μm 100 Å 2.1×150 mm. For each analytical separation approximately 0.5 OD₂₆₀ of crude DNA was injected as a 20-50 μL solution in Millipore water. Detection was carried out using a diode-array detector, monitoring absorbance at 260 nm. Retention times and yields (obtained from peak area integration) for the products are summarized in Table 2.

TABLE 2
HPLC data summary for all compounds.

| Molecule | Yield/% | Retention time/min. |
|---|---|---|
| HE₁-DNA | 89 | 13.0 |
| HE₂-DNA | 85 | 16.0 |
| HE₃-DNA | 86 | 17.7 |
| HE₄-DNA | 84 | 18.9 |
| HE₅-DNA | 83 | 19.7 |
| HE₆-DNA | 83 | 20.5 |
| HE₁₂-DNA | 74 | 22.9 |
| (HE-HEG)₆-DNA | 51 | 18.6 |
| (HE₂-HEG₂)₃-DNA | 62 | 19.4 |
| (HE₃-HEG₃)₂-DNA | 56 | 19.9 |
| HE₆-HEG₆-DNA | 67 | 21.0 |
| HEG₆-HE₆-DNA | 78 | 19.8 |

The yields presented are the percentage of desired product with reference to the total DNA cleaved from the solid support (capped failure sequences).
The retention time of the DNA control was 9.5 minutes.

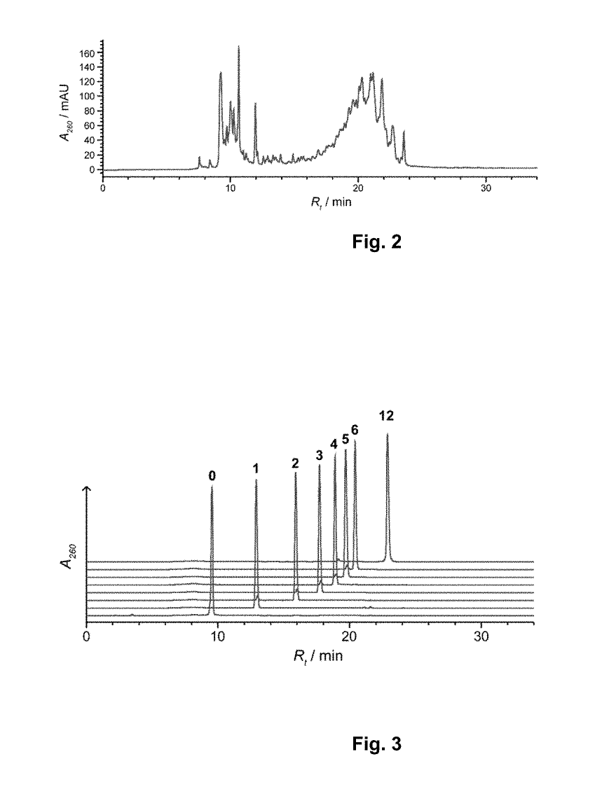

The process defined herein, therefore allows the synthesis of monodisperse conjugates in good yields. This is in contrast to a conjugate obtained by random synthesis. For example, FIG. 2 is illustrating the copolymer obtained by performing 12 couplings at the 5′ terminus of the 19mer oligonucleotide using a 1:1 (w/w) mixture of DMT-hexaethyloxy Glycol and DMT-dodecane-diol amidites using the aforementioned synthetic protocol. The DMT⁺ response revealed high coupling efficiency of all 12 random units. The HPLC trace shows a major distribution of products centered on the retention times seen for the 12 unit sequenced products.

Since the coupling efficiency remains excellent (>97%), it is surely possible to generate much longer polymers, however in this work we chose to stop at twelve units, due to the ease of division into smaller blocks for sequence definition (vide infra).

### Conjugates Comprising HE Blocks to the 5′ Terminus of the Oligonucleotide:

Analysis of the crude mixtures by reverse-phase HPLC (Table 2) revealed a narrow product distribution where the target molecule constituted 74%-89% of the synthesised DNA, a much greater yield than generally found in couplings of full-length polymers to DNA strands. Reverse-phase HPLC analysis of the products (FIG. 3) revealed increasing retention time with number of HE blocks, consistent with an increase in hydrophobicity with each addition of HE blocks to the 5′ terminus of the oligonucleotide.

### Conjugates Comprising HE(HEG) Blocks:

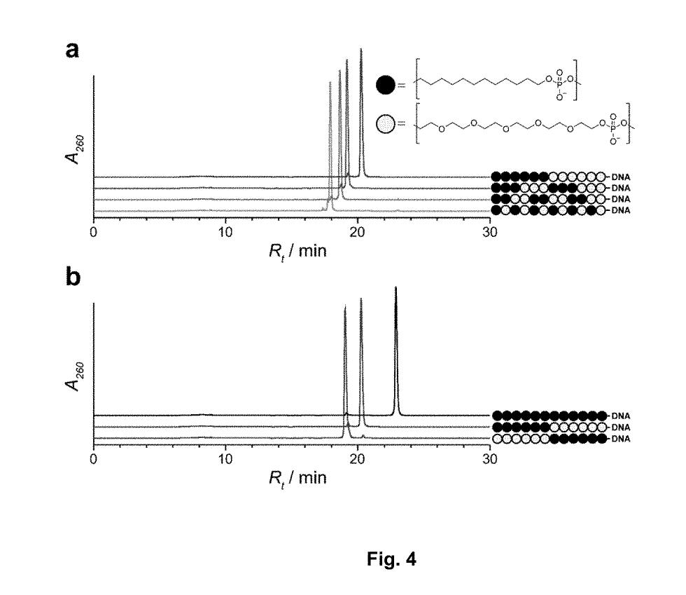

The shortest retention time in the HPLC column is observed for the alternating pattern (HE-HEG)₆-DNA, thus it is the least hydrophobic pattern. The longest retention time, and therefore most hydrophobic pattern, was seen for the construct containing six blocks of HE together, the HE₆-HEG₆-DNA conjugate. This suggests a dependency on the sequence of the two units for the overall hydrophobicity, and indeed a gradual increase in retention time is observed as the hydrophobic block size increases from one to six units (FIG. 4a). It is reasonable that the higher the number of adjacent HE units present in the 5′ modification, the more hydrophobic the resulting product. This may be due to the fact that a minimal number of adjacent HE are needed to create a hydrophobic pocket that is available for interaction with the stationary phase, whereas in alternating (HE-HEG)₆-DNA the hydrophilic groups flank each HE unit and only the terminal moiety is available for strong interaction with the stationary phase.

Another variable in the block pattern that affects the hydrophobic behaviour is the position of the HE and HEG blocks with respect to the hydrophilic oligonucleotide; this is evident when comparing the molecules HE₆-HEG₆-DNA and HEG₆-HE₆-DNA (FIG. 4b). In terms of the amphiphilicity of the different block patterns, HE₆-HEG₆-DNA is more akin to a typical amphiphile with a distinct hydrophilic DNA-HEG block and a hydrophobic HE terminus. The bola-amphiphilic HEG₆-HE₆-DNA may adopt a structure in which the oligonucleotide and the HEG blocks are more capable of shielding the HE portion from the aqueous medium.

### MALDI-MS Characterization

The matrix solution was comprised of a 1:1 mixture of ammonium citrate solution (50 mM, water) and 2,4,6-trihydroxyacetophenone solution (sat., MeCN) solution. In each case 1 μL of a 50-100 μM solution of HPLC purified DNA in Millipore water was mixed with 1 μL of matrix solution. Between 0.5-1 μL of this final solution was spotted on an AnchorChip™ and solvents were removed by air drying prior to mass determination. Analysis was carried out in linear negative mode. In each case, the masses observed for the DNA conjugates matched well with the calculated values.

TABLE 3
MALDI-MS Calculated and experimental m/z values
for all DNA conjugates synthesized.

| Molecule | Calculated m/z ([M − H]⁻) | Found m/z ([M − H]⁻) |
|---|---|---|
| HE₁-DNA | 6027.13 | 6024.84 |
| HE₂-DNA | 6292.28 | 6288.06 |
| HE₃-DNA | 6556.43 | 6551.91 |
| HE₄-DNA | 6820.58 | 6815.98 |
| HE₅-DNA | 7084.73 | 7080.73 |
| HE₆-DNA | 7348.87 | 7344.72 |
| HE₁₂-DNA | 8933.77 | 8931.10 |
| (HE-HEG)₆-DNA | 9413.62 | 9408.68 |
| (HE₂-HEG₂)₃-DNA | 9413.62 | 9410.85 |
| (HE₃-HEG₃)₂-DNA | 9413.62 | 9412.72 |
| HE₆-HEG₆-DNA | 9413.62 | 9412.00 |
| HEG₆-HE₆-DNA | 9413.62 | 9410.99 |

### LC-ESI-MS Characterization

The oligonucleotides were analyzed by LC-ESI-MS in negative ESI mode. Samples were run through an Acclaim RSLC 120 C18 column (2.2 μM 120 Å 2.1×50 mm) using a gradient of 98% mobile phase A (100 mM 1,1,1,3,3,3-Hexafluoro-2-propanol and 5 mM Triethylamine in water) and 2% mobile phase B (Methanol) to 40% mobile phase A and 60% mobile phase B in 8 minutes. The data was processed and deconvoluted using the Bruker DataAnalysis software version 4.1

TABLE 4
ESI-MS characterization and percent yields of conjugates.

| | | Calculated | Mass |
|---|---|---|---|
| Conjugate^{[a]} | Yield [%]^{[b]} | mass [Da] | found [Da] |
| HE₁ | 89 | 6029.14 | 6029.13 |
| HE₂ | 85 | 6293.29 | 6293.29 |
| HE₃ | 86 | 6557.44 | 6557.45 |
| HE₄ | 84 | 6821.59 | 6821.61 |
| HE₅ | 83 | 7085.74 | 7085.69 |
| HE₆ | 83 | 7349.88 | 7349.83 |
| HE₁₂ | 74 | 8934.78 | 8934.68 |
| (HE-HEG)₆ | 51 | 9414.63 | 9414.60 |
| (HE₂-HEG₂)₃ | 62 | 9414.63 | 9414.58 |
| (HE₃-HEG₃)₂ | 56 | 9414.63 | 9414.58 |
| HE₆-HEG₆ | 67 | 9414.63 | 9414.58 |
| HEG₆-HE₆ | 78 | 9414.63 | 9414.60 |

^{[a]}Attached to the 5′ terminus of DNA sequence TTTTTCAGTTGACCATATA (SEQ ID NO: 3)
^{[b]}Obtained from HPLC peak area integration

### Dynamic Light Scattering and Hydrodynamic Radii

The conjugates described herein can be further characterized with regard to their self-assembly behavior. To probe the self-assembly in solution, dynamic light scattering (DLS) was used to determine the presence, and hydrodynamic radius, of micellar aggregates.

Dynamic Light Scattering (DLS) experiments were carried out using a DynaPro™ Instrument from Wyatt Technology. A cumulants fit model was used to confirm the presence and determine the size of a monomodal population of micellar aggregates. Sterile water and TAMg buffer were filtered using a 0.45 μm nylon syringe filter before use for DLS sample preparation. All measurements were carried out at 25° C.

It was found that the HEₓ-DNA containing five or less HE units existed as discrete molecules at 10 μM in magnesium containing buffer, indicated by poor scattering intensities. However, for HE₆-DNA strong scattering correlating to a sphere with hydrodynamic radius (Rₕ) of 6.5±0.4 nm was observed, Table 2. This size correlates with DNA-based spherical micelles and is consistent with the tight packing of the HE chains in a hydrophobic core with a charged corona made up of DNA. Assuming a linear DNA geometry (6.1 nm), this result suggests that the HE₆ chain (1.9 nm/unit if stretched) is folded on itself multiple times, potentially adopting a ‘concertina’ structure analogous to that of phospholipid bilayers. HE₁₂-DNA under the same conditions revealed an Rₕ of 11.3±0.1 nm, implying that the HE₁₂ chain is more extended.

TABLE 5
Hydrodynamic Radii for DNA-conjugate micelles by DLS.

| | Single-stranded Rₕ | Double-stranded Rₕ |
|---|---|---|
| Conjugate | [nm] | [nm] |
| HE₆ | 6.5 ± 0.4 | 5.6 ± 0.1 |
| HE₁₂ | 11.3 ± 0.7 | 11.3 ± 0.1 |
| HE₆-HEG₆ | 9.7 ± 0.1 | 10.0 ± 0.2 |

All samples were measured in at least triplicate and the mean values for hydrodynamic radii are shown below. Error margins were derived from the standard deviation of the multiple measurements.

To investigate the effect of block pattern on the amphiphilic self-assembly in solution, DLS measurements were carried out for the HE/HEG series. Samples were annealed in TAMg buffer at 10 μM, and measured at 25° C. Analysis of the DLS data revealed that the trends in hydrophobicity observed by HPLC were amplified under self-assembly conditions. The strands with polymer sequences (HE-HEG)₆, (HE₂-HEG₂)₃, and (HE₃-HEG₃)₂ did not exhibit self-assembly at this concentration. However strong scattering was seen for HE₆-HEG₆-DNA with an associated Rₕ of 9.7±0.9 nm, although not for HEG₆-HE₆-DNA. Firstly, the DLS observations lend further weight to the hypothesis that a minimum block size is required to exhibit significant hydrophobic character, as blocks below six units do not exhibit aggregation under these conditions. Furthermore, the effect of the positioning of the blocks with respect to the oligonucleotide portion also appears to be a major factor in the self-assembly: the terminal HE₆ results in micellization, whereas a central HE₆ block prevents aggregation.

### Melting Temperatures and Electrophoretic Mobility Assays

### Melting:

The hybridization of the DNA amphiphiles to their complement strands was confirmed by measurement of the thermal denaturation of the duplexes by UV absorption. For each experiment equimolar amounts of DNA amphiphile and complement strand (Acomp, see Table 1) were combined in TAMg buffer to give a final volume of 150 μL with a duplex concentration of 2.5 μM. Samples were thermally annealed (95 to 4° C., 4 hours) before transfer to a quartz cuvette. Absorbance at 260 nm was monitored over the appropriate temperature range.

Typical sigmoidal melting curves were obtained for the hybridization of the complementary strand Acomp to DNA control, discrete (HE-HEG)₆-DNA and HE₁₂-DNA micelles. This curves showed that in both the case of discrete molecules and self-assembled aggregates the oligonucleotide section is still available for hybridization, with little change in melting temperature (3° C., between DNA and HE₁₂-DNA).

### Electrophoretic Gel

Native Polyacrylamide Gel Electrophoresis (PAGE) was carried out at room temperature for 2 hours at a constant voltage of 250V. Sample loading was 0.01 OD₂₆₀ DNA per lane. Single-stranded samples were annealed (95 to 4° C., 4 hours) prior to loading on the gel to promote uniform assembly. Double-stranded samples were similarly annealed (with Acomp, see Table 1) to provide 25 μL of 10 μM duplex in 1×TAMg. The DNA bands were visualized by incubation with GelRed™.

On the gel electrophoresis assay, each lane was loaded with one conjugate (i.e. HE₁-DNA, HE₂-DNA, HE₃-DNA, HE₄-DNA, HE₅-DNA, HE₆-DNA, HE₁₂-DNA, (HE-HEG)₆-DNA, (HE₂-HEG₂)₃-DNA, (HE₃-HEG₃)₂-DNA, (HE)₆(HEG)₆-DNA, HEG₆-HE₆-DNA). The mobility of the micelles formed by HE₆-DNA, HE₁₂-DNA and HE₆-HEG₆-DNA correspond well with the hydrodynamic radii determined by DLS.

Thermal denaturation and gel electrophoresis concluded that all of the HEₓ-DNA molecules retained their ability to hybridize to a complementary DNA strand, highlighting the orthogonal nature of these differing modes of intermolecular self-assembly.

Thermal denaturation and gel electrophoresis of HExHEGy block conjugates confirmed the orthogonality of these assembly mechanisms. Gel bands were sharp and mobilities decreased with increasing hydrophobicity; conjugates HE₁₂-DNA and HE₆HEG₆-DNA showed non-penetrating bands, consistent with their micellar aggregation. Thermal denaturation temperatures for the double-stranded DNA-polymer conjugates were similar to that of the parent DNA duplex.

### Fluorescence of Encapsulated Nile Red

To further show that the behavior of the conjugates was in line with block copolymer self-assembly, encapsulation of guest molecules within the hydrophobic core of the micelles was performed using Nile Red, a fluorescent dye which displays significant fluorescence in hydrophobic media, but negligible emission in aqueous solution.

DNA-conjugates (10 μM) in TAMg buffer (60 μL) were thermally annealed (95 to 4° C., 4 hours). The samples were transferred to glass vials and 1.5 μL of Nile Red solution (0.1 μM, acetone) was added, to give a final Nile Red concentration of 2.5 μM. The samples were vortexed briefly, sealed and incubated overnight at room temperature in the absence of light. Fluorescence spectra were recorded at room temperature in a 50 μL quartz cuvette using an excitation wavelength of 535 nm, and monitoring emission between 560 and 750 nm, with excitation and emission slit widths both set at 10 nm.

An increase in fluorescence was observed with increasing number of HE units in a 10 μM aqueous solution of the polymer-DNA conjugates, showing the expected dependence of the self-assembled macromolecular structure on the component molecular structure.

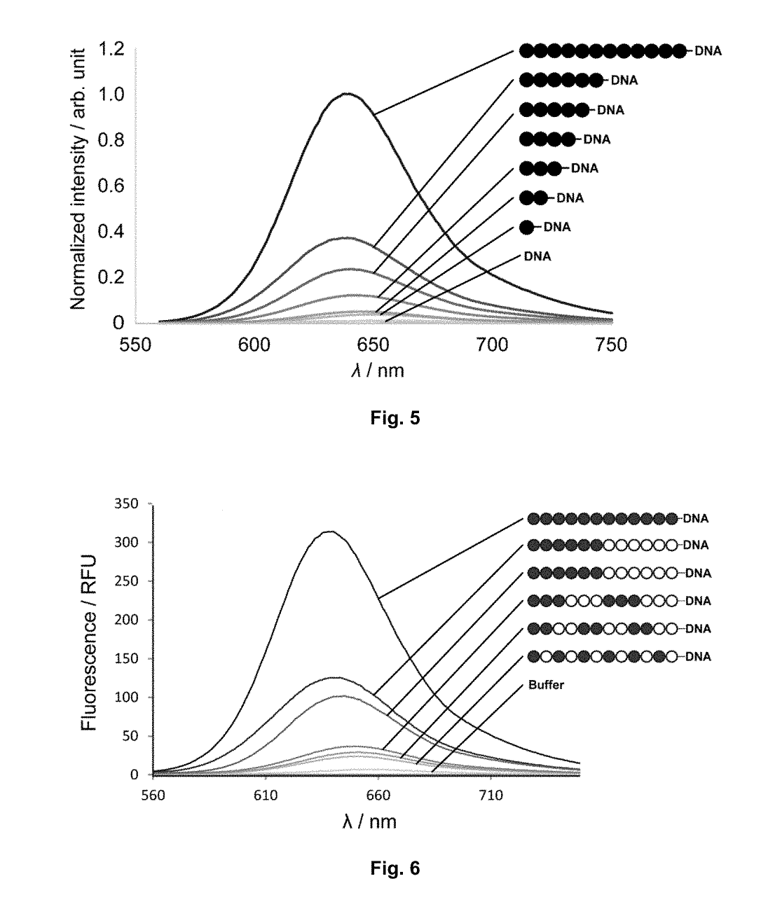

The sequence-specific self-assembly was also assessed using encapsulation of Nile Red (FIG. 5). In this case a small increase in fluorescence was seen as the block size increased from one to three HE units, consistent with a degree of intramolecular collapse of adjacent HE units, generating progressively larger hydrophobic domains as the block size increases, akin to beads on a string. At a block size of six, both of the DNA-sequence polymers displayed a similar level of Nile Red fluorescence, although that of HEG₆-HE₆-DNA was slightly lower (FIG. 6). A small, but repeatedly measurable, red shift of the maximum emission from the bola-amphiphile (640 nm) with respect to the linear amphiphile (645 nm) was also observed, which is indicative of Nile Red in a more polar environment P. Greenspan, S. D. Fowler, J. Lipid Res. 1985, 26, 781-789). This is consistent with a intermolecular spherical micelle formed by HE₆-HEG₆-DNA, and a (still moderately sized) intramolecular hydrophobic domain being formed in HEG₆-HE₆-DNA, resulting in a lower volume-to-surface area ratio, and more exposure of the dye to the solvent.

### Solid-Phase Synthesis of HEₓ-RNA Conjugates

RNA synthesis was performed on a 1 μmole scale, starting from a universal 1000 Å LCAA-CPG solid-support. Coupling efficiency was monitored after removal of the dimethoxytrityl (DMT) 5′-OH protecting groups. DMT-dodecane-diol (cat.# CLP-1114) phosphoramidite was purchased from Chemgenes. DMT-dodecane-diol phosphoramidite was dissolved (0.1M, anhydrous acetonitrile under a nitrogen atmosphere in a glove box (<0.04 ppm oxygen and <0.5 ppm trace moisture). For the addition of each RNA nucleoside phosphoramidites and DMT-dodecane-diol phosphoramidite, extended coupling times of 6 and 5 minutes were used, respectively. The coupling activator used was 0.25M 5-(Ethylthio)tetrazole in anhydrous acetonitrile. Removal of the DMT protecting group was carried out using 3% dichloroacetic acid in dichloromethane on the DNA synthesizer. Completed syntheses were cleaved from the solid support and deprotected in 28% aqueous ammonium hydroxide solution for 16-18 hours at room temperature. The crude product solution was separated from the solid support and concentrated under reduced pressure at 60° C. This crude solid was re-suspended in 150 μL of a desilylation solution containing triethylamine, N-methylpyrrolidone, and triethylaminetrihydrofloride (3:2:1.5) and heated to 65° C. for 2 hours, to remove the 2′-OH tert-butyldimethylsilyl protecting groups. This desilylation step is then quenched by the addition of 100 μL of 3 M sodium acetate (pH 5.5) and vortexed. The RNA is precipitated by addition of 1 mL cold butanol and left 30 minutes at −20° C. The precipitate was collected as a pellet through centrifugation and the butanol is removed. The pellet is washed a second time with butanol, and dried under reduced pressure at 60° C. This crude product of RNA or HE-RNA conjugate was resuspended in DEPC-treated sterile water and quantified by absorbance at 260 nm prior to purification by either PAGE or HPLC.

TABLE 6
RNA and HE-RNA conjugates.

| | | SEQ |
|---|---|---|
| Molecule | Sequence (5′-xx-3′) | ID NO: |
| Luc-g | uuaauuaaagacuucaagcGG | 4 |
| Luc-p | gcuugaagucuuuaauuaaTT | 5 |
| Luc-p-HE₁₂ | gcuugaagucuuuaauuaaTTAAAAADDDD | 6 |
| | DDDDDDDD | |
| HE₁₂-Luc-p | DDDDDDDDDDDDgcuugaagucuuuaauua | 7 |
| | aTT | |
| Luc-g-HE₁₂ | uuaauuaaagacuucaagcGGAAAAADDDD | 8 |
| | DDDDDDDD | |
| ApoB-g | auugguauucagugugaugac*a*c | 9 |
| ApoB-p | gucaucacacugaauaccaa*u | 10 |
| HE₁₂-ApoB-g | DDDDDDDDDDDDauugguauucagugugau | 11 |
| | gac*a*c | |

(D = residue obtained from reaction of DMT-dodecane-
diol amidite
Lower case letters indicate RNA, uppercase letters
indicate DNA, bold letters indicate 2′-OMe RNA
nucleotides and * represents phosphorothioate linkages.)

### PAGE Purification of HEₓ-RNA Conjugates

The crude products were purified on 20% polyacrylamide/8M urea PAGE at constant current of 30 mA for 1.5 hours (30 min at 250V followed by 1 hr at 500V), in TBE buffer. The product bands were excised under UV shadowing on silica plates, and the gel pieces were crushed and incubated in 12 mL of sterile DEPC-treated water at 65° C. for 12-16 hours. Samples were then dried to ca. 1.5 mL, desalted using size exclusion chromatography (Sephadex G-25), and quantified by absorbance at 260 nm.

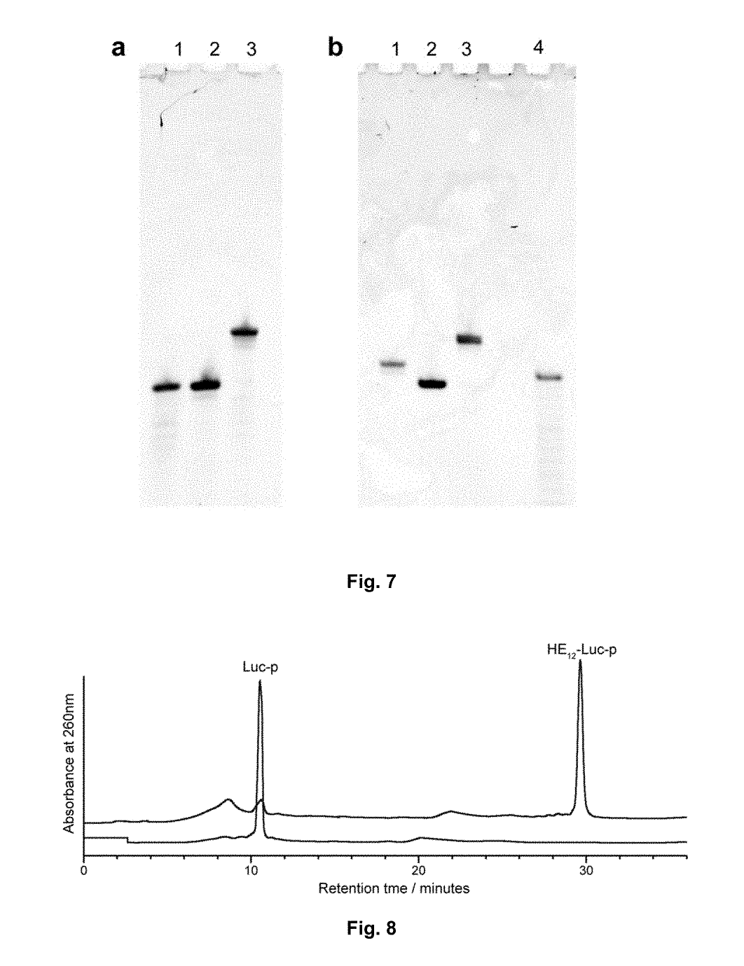

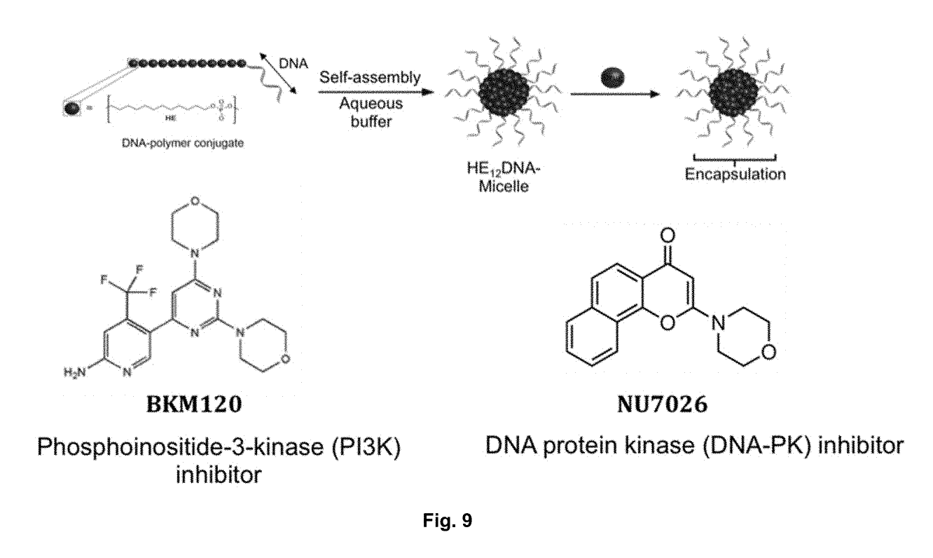

FIG. 7 represents the gel electrophoresis of RNA conjugates described above: a) FF Luciferase targeted siRNA; Lane 1: Luc-g, Lane 2: Luc-p, Lane 3: HE12-Luc-p. b) Apolipoprotein B targeted siRNA; Lane 1: ApoB-g, Lane 2: ApoB-p, Lane 3: HE12-ApoB-p, Lane 4: ApoB-p-11. Reduced mobility in lanes a3, b3 and b4 in contrast to the unmodified controls in other lanes indicates coupling of the desired modifications.

### HPLC Analysis of Crude HEx-RNA Conjugates

The crude products were analyzed by reverse-phase HPLC to confirm the attachment of the hydrophobic HE₁₂ section to the RNA portion. Solvents (0.22 μm filtered): 50 mM Triethylammonium acetate (TEAA) buffer (pH 8.0) and HPLC grade acetonitrile Elution gradient: 3-50% acetonitrile over 30 minutes at 60° C. Column: Hamilton PRP-C18 5 μm 100 Å 2.1×150 mm. For each analytical separation approximately 0.5 OD₂₆₀ of crude RNA was injected as a 20-50 μL solution in sterile DEPC-treated water. Detection was carried out using a diode-array detector, monitoring absorbance at 260 nm.

TABLE 7
Summary of HPLC data for RNA conjugates

| Molecule | Retention time/min. |
|---|---|
| Luc-g | 10.6 |
| Luc-p | 10.1 |
| HE₁₂-Luc-p | 29.6 |
| HE₁₂-ApoB-g | 28.8 |
| ApoB-g-NC16₁ | 26.0 |

FIG. 8. Is a representative HPLC traces of HE12-RNA versus unmodified control.

### LC-MS Analysis of HE₁₂-RNA Conjugate

The oligonucleotides were analyzed by LC-ESI-MS in negative ESI mode. Samples were run through an Acclaim RSLC 120 C18 column (2.2 μM 120 Å 2.1×50 mm) using a gradient of 98% mobile phase A (100 mM 1,1,1,3,3,3-Hexafluoro-2-propanol and 5 mM Triethylamine in water) and 2% mobile phase B (Methanol) to 40% mobile phase A and 60% mobile phase B in 8 minutes. The data was processed and deconvoluted using the Bruker DataAnalysis software version 4.1.

TABLE 8
LC-MS data comparing unmodified RNA with HE₁₂-Luc-p.

| | Calculated mass | Mass found |
|---|---|---|
| Conjugate^{[a]} | [Da] | [Da] |
| Luc-g | 6669.94 | 6670.00 |
| Luc-p | 6613.88 | 6613.94 |
| HE₁₂-Luc-p | 9787.68 | 9787.80 |

### Encapsulation of BKM120 and NU7026:

BKM120 was prepared as a 10 mM working solution in chloroform. Loading of the structures was achieved by adding 20 μL of BKM120 to a glass vial, followed by solvent evaporation in open air to achieve a thin drug film. HE₁₂-DNA micelles were pre-assembled in 1×TAMg (100 μL at 10 mM) and subsequently added to the drug film. The final solution was 100 μL with excess BKM120 (2 mM). The mixture was vortexed heavily to allow re-suspension of the drug molecules, and was incubated overnight at room temperature. Alternatively, drug loading could also be obtained by a different method. In this protocol, 20 μL of the chloroform solution of BKM120 was added to 100 μL of stirred aqueous solution of pre-assembled micelles in 1×TAMg, forming an o/w emulsion. The o/w emulsion was incubated overnight at room temperature in an open atmosphere, allowing chloroform evaporation.

### Purification of BKM120 Loaded HE₁₂-AT Micelles

Removal of free BKM120 was achieved by preparative centrifugation (15,000×g, 4° C., 1 hour) between 2-4 times or size exclusion chromatography Illustra MicroSpin G-25 Columns (GE Healthcare), although the recovery of HE₁₂-AT micelles is not high using the manufacturer's protocol.

RP-HPLC and UV-Vis spectroscopy were used to determine the amount of BKM120 loaded in the DNA micelles, For HPLC, 60 μL of the purified supernatant was injected into a Hamilton PRP-1 5 μm 100 Å 2.1×150 mm column. The solvents used were 50 mM Triethylammonium acetate (TEAA) buffer (pH 7.8) and HPLC grade acetonitrile. Typical retention times for the products are 27.4 minutes (DNA-polymer conjugate) and 28.733 minutes (BKM120) at 260 nm detection channel. The products were also detected using a drug-only channel at 320 nm (BKM 120 maximum absorption peak).

For UV-Vis spectroscopy experiments, 20 μL of the purified supernatant was mixed with 80 μL of DMSO, and the absorbance (200 nm-500 nm) was measured. The presence of BKM120 was monitored by the absorbance at 320 nm using a Biotek Synergy HT well-plate spectrometer. The same encapsulation, purification and detection protocol was followed for NU7026.

HPLC results indicate the co-elution of BKM120 and HE₁₂-DNA conjugates. These results suggest that BKM120 remains encapsulated following purification. The amount of incorporated BKM 120 was calculated by measuring HPLC peak areas of BKM 120 and DNA. Standard curves of known concentrations of DNA and BKM120 were used to determine the loading capacity of the HE₁₂-DNA micelle.

HPLC data indicates that efficient encapsulation of BKM120. These results suggest that 8 BKM120 molecules were incorporated per HE₁₂-DNA conjugate unit.

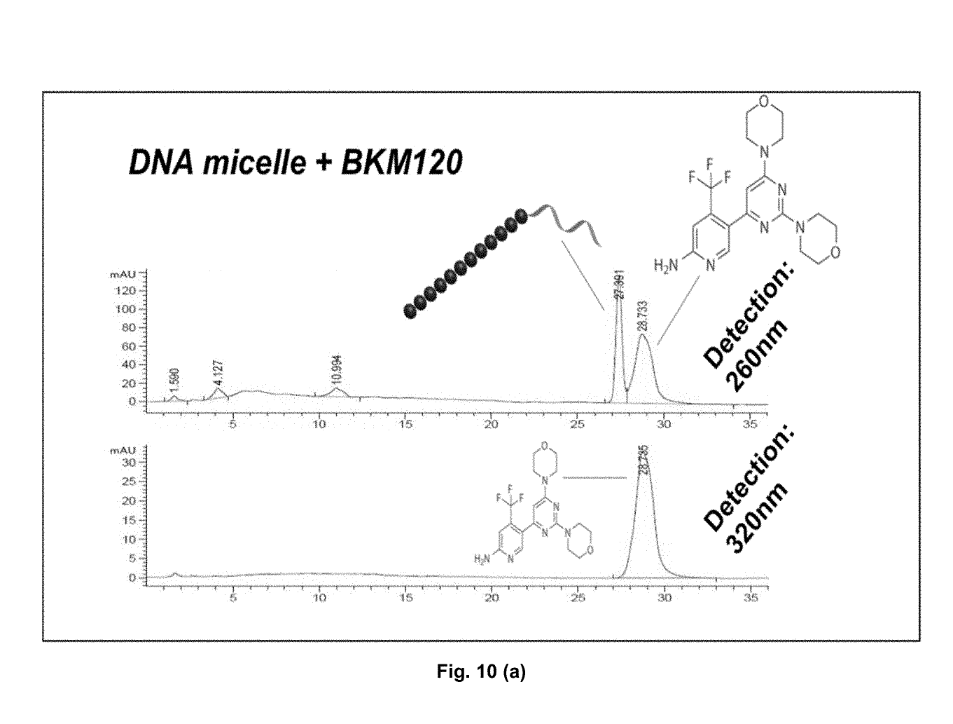

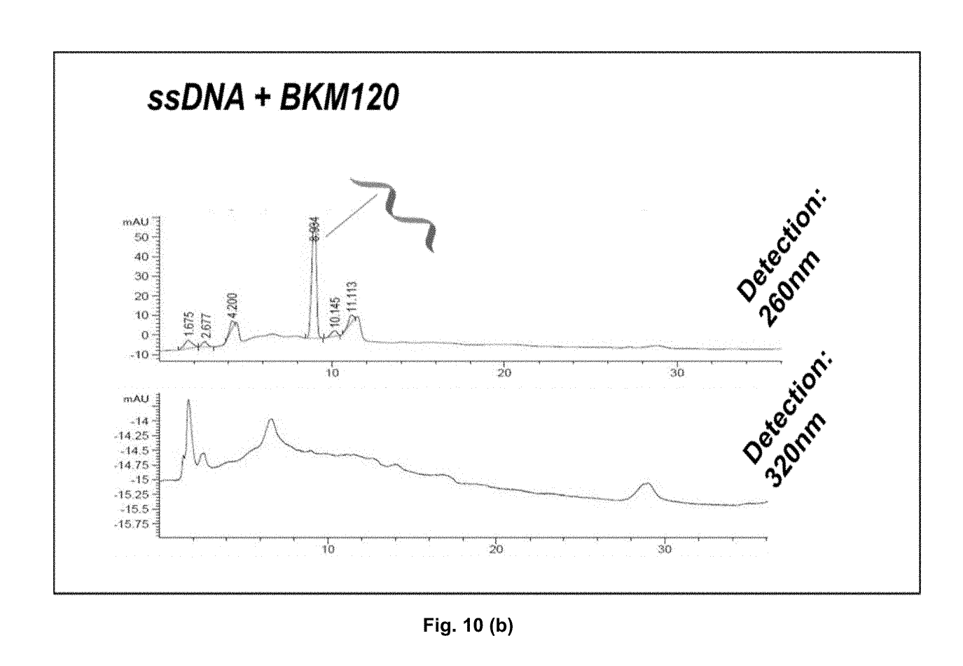

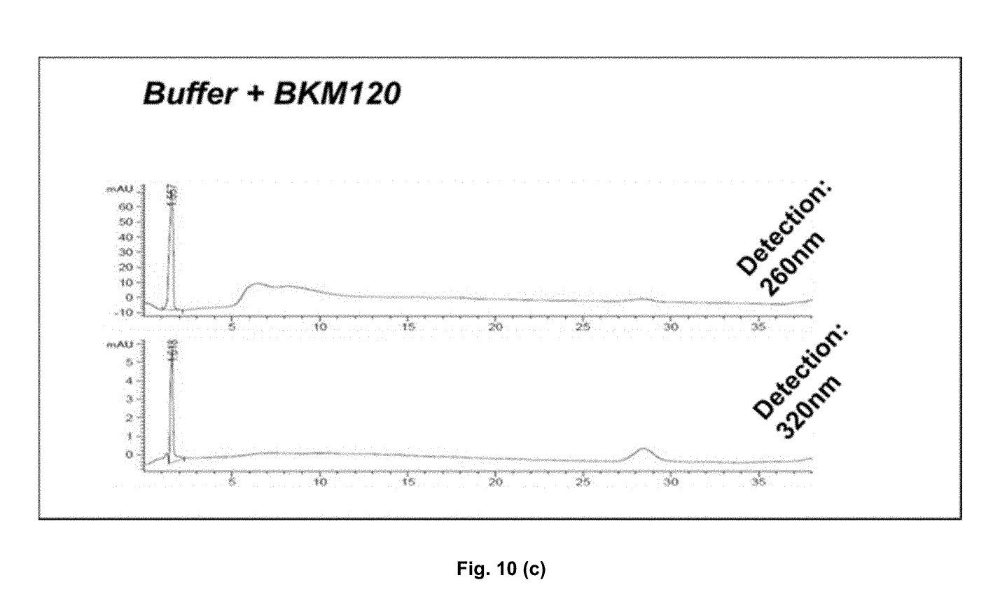

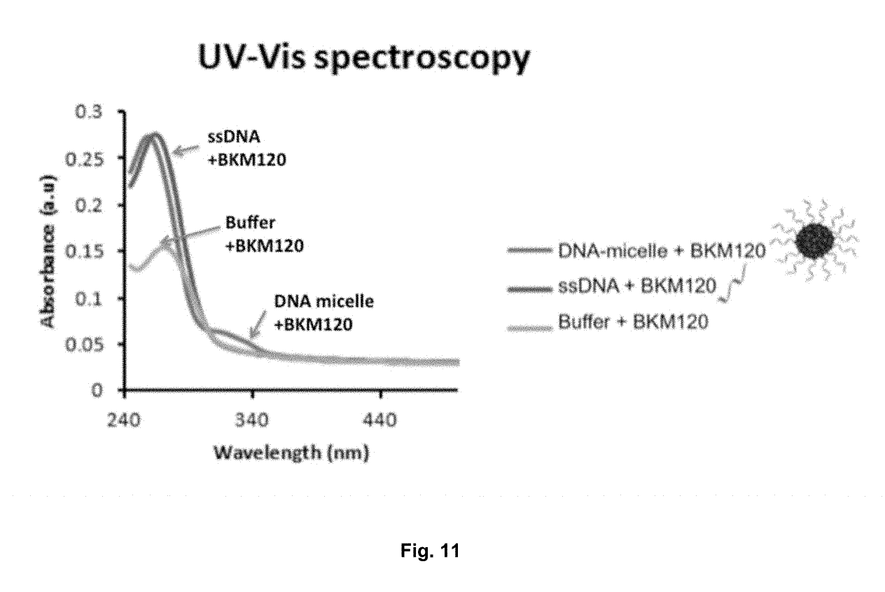

FIG. 10. are HPLC traces of size exclusion chromatography purification products. a) Purified HE₁₂-DNA micelle-BKM120 mixture. b) Purified ssDNA control-BKM120 mixture. The ssDNA sequence is identical to the DNA portion of the DNA-polymer conjugate. c) Purified Buffer control-BKM120 mixture.

Drug encapsulation was further confirmed by UV-Vis spectroscopy by measuring the diagnostic peak of BKM120 absorbance at 320 nm. FIG. 11 illustrates the absorbance at 320 nm compared between purified mixtures of HE₁₂-DNA micelle-BKM120, ssDNA-BKM120 and Buffer-BKM120. The peak at 320 nm was also used to further confirm the BKM120 loaded concentration determined by HPLC.

### Characterization of BKM120-Loaded HE₁₂-DNA Micelles:

### Gel Mobility Shift Assays

Agarose gel electrophoresis was used to characterize the BKM120-loaded HE₁₂-DNA micelles. In each case, 2.5% AGE was carried out at 4° C. for 2.5 hours at a constant voltage of 80V. Typical sample loading is 15 picomoles with respect to the DNA, per lane (1.5 μL of 10 μM DNA). BKM120-loaded HE₁₂-DNA micelles were compared to an empty HE₁₂-DNA micelle control and ssDNA. A gel analysis reflected the maintained structural integrity of the drug-loaded micelles, and the absence of any degradation products.

### Dynamic Light Scattering

Dynamic light scattering (DLS) experiments were carried out using a DynaPro™ Instrument from Wyatt Technology. A cumulants fit model was used to confirm the presence and determine the size the BKM120-loaded HE₁₂-DNA micelles.

Sterile water and 1×TAMg buffer were filtered using a 0.45 μm nylon syringe filter before use in DLS sample preparation. All measurements were carried out in triplicate at 25° C.

DLS data reveals a highly monodisperse population of BKM120-loaded HE₁₂-DNA micelles. The hydrodynamic radius of the drug-loaded micelles was calculated to be R_{H}=11.2±0.2 nm.

### Atomic Force Microscopy

Dry AFM was carried out using a MultiMode8™ SPM connected to a Nanoscope™ V controller (Bruker, Santa Barbara, Calif.). All images were obtained using tapping mode in air with AC160TS cantilevers (Nominal values: Tip radius—9 nm, Resonant frequency—300 kHz, Spring constant—42 N/m) from Asylum Research. Samples were diluted to 1 μM in TAMg buffer and 4 μL of this solution was deposited on a freshly cleaved mica surface (ca. 7×7 mm) and allowed to adsorb for 1-2 seconds. Then 50 μL of 0.22 μm filtered Millipore water was dropped on the surface and instantly removed with filter paper. The surface was then washed with a further 200 μL of water and the excess removed with a strong flow of nitrogen. Samples were dried under vacuum for 15-30 minutes prior to imaging.

The spherical morphology, monodispersity and relative size of BKM120-loaded micelles were confirmed by AFM. The average height of 1.8 nm and width of 26 nm are consistent with the obtained DLS results. This flat and slightly wide morphology is due to deformation of the micelles upon surface adsorption and drying.

### Transmission Electron Microscopy

Samples (2 μL at 0.5 μM w.r.t. total DNA) were deposited on carbon film coated copper EM grids for one minute, followed by blotting off the excess liquid with the edge of a filter paper, and washing three times with 20 μL of water, before drying under vacuum. The samples were imaged using a Tecnai 12 microscope (FEI electron optics) equipped with a Lab6 filament at 120 kV. Images were acquired using a Gatan 792 Bioscan 1 k×1 k Wide Angle Multiscan CCD Camera (Gatan Inc.). Contrast was adjusted automatically—note that in the presence of any high-contrast foreign matter, this results in the micelles being almost invisible. Images were analyzed using ImageJ, which required manually setting threshold levels and placing limits on the size and circularity of features to ensure correct particle picking. The area values obtained were converted into radii (for comparison with DLS), making the assumption that the features are circular, which can be readily validated by eye. Images were acquired using a FEI Tecnai G2 F20 operating at 120 kV equipped with a Gatan Ultrascan 4000 4 k×4 k CCD Camera System Model 895.

The relative size and spherical morphology of drug loaded micelles was further confirmed by TEM. The average diameter of BKM120-loaded micelles was calculated d=24.6 nm. These results are consistent for the DLS and AFM size analysis. This slight wider morphology of the drug-loaded micelles could be due to structure deformation upon surface adsorption and drying

### Drug Release Kinetics

An aliquot of the BKM120-loaded micelles was taken immediately after purification. The initial amount of encapsulated drug (Absorbance t₀) was determined by measuring the drug absorbance at λmax=320 nm. Following the initial measurement, BKM120-loaded micelles were incubated at room temperature for 12 hour intervals. After each incubation period, samples were re-purified by preparative centrifugation (15,000×g at 4° C.). The amount of drug released was estimated from the measurement at each sampling point at λmax=320 nm. The drug concentration could be directly calculated from the measured absorbance. The amount of drug released was calculated from the amount of drug initially present in the micelles and the amount of drug retained in the micelles at each sampling point. The percent drug release was calculated using the equation given below:

$$\% BKM 120 encapsulated = ( \frac{Absorbance ( t )}{Absorbance ( {t}_{0} )} ) \times 100$$

Preliminary results indicate over 70% BKM120 retention over a span of 6 days. The first 48 hours are characterized by a fast release of the drug, followed by a slower release until reaching a plateau ˜72 hours.

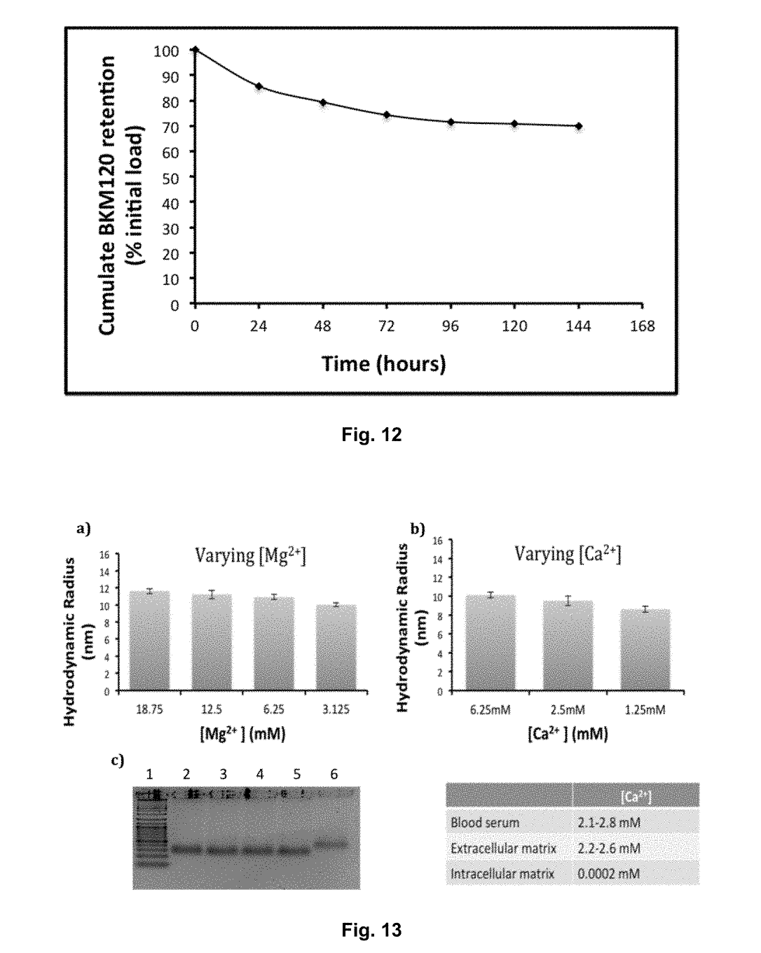

In the measurements described above and in FIG. 12, no account was taken for the DNA absorbance. Therefore, any DNA loss during purification could have correlated to a lower drug absorbance signal that was unaccounted for, however this is expected to be minimal.

### Micelle Stability:

HE₁₂-DNA micelles were assembled under thermal annealing in different ionic concentrations. In general, each reaction mixture contained 20 μL of 10 μM DNA mixed with Tris-acetic acid buffers of different ionic compositions (18.75-3.125 mM final Mg²⁺ and 6.25-1.25 mM final Ca²⁺ concentration). Following thermal annealing, dynamic light scattering was performed on the 20 μL samples to determine the average size and mono-dispersity of the resulting products.

In another set of experiments, Cy3-labeled HE₆-DNA micelles were thermally annealed with varying concentrations of Dulbecco's Phosphate Buffered Saline purchased from Life Technologies. The concentrations of DPBS were chosen to be compatible with buffer requirements for in vivo work.

FIG. 13 relates to in vitro micelle stability studies in physiologically relevant conditions FIG. 13(d). Dynamic light scattering and agarose gel electrophoresis were used to determine the structural stability of HE₁₂-DNA micelles under different buffer and ionic conditions. DLS measurements with a) varying magnesium concentrations, b) varying calcium concentrations. c) Agarose gel elecltrophoresis of HE6-DNA micelles assembled in 1×DPBS buffer. Lane 1—DNA ladder, lane 2—Cy3-HE6-DNA micelles in 2×DPBS, lane 3—Cy3-HE₁₂-DNA micelles in 1×DPBS, Cy3-HE6-DNA micelles in 0.5×DPBS, lane 5—Cy3-HE6-DNA micelles in 0.25×DPBS, lane 6—HE6-DNA micelle control in 1×TAMg.

Structural characterization shows great stability of DNA-polymer micelles in various buffers mimicking physiological conditions. As stabilizing cations are required for the self-assembly of the DNA-polymer conjugates, in vitro work on changing the ion concentrations (Magnesium, Calcium) to resemble what's found in blood serum and extra-cellular matrix, reflects the maintained structural integrity of the micellar structures. Assembly in DPBS further indicates that these systems are compatible with physiological systems.

### Nile Red Encapsulation:

Nile Red was prepared as a 10 mM working solution in acetone. Loading of the structures was achieved by adding 20 μL of Nile Red to a glass vial, followed by solvent evaporation in open air to achieve a thin drug film. HE₁₂-DNA micelles were pre-assembled in 1×TAMg (100 μL at 10 mM) and subsequently added to the drug film. The final solution was 100 μL with excess Nile Red (2 mM). The mixture was vortexed heavily to allow re-suspension of the drug molecules, and was incubated overnight at room temperature. The mixture was purified by preparative centrifugation (15,000×g, 4° C., 1 hour) between 2-4 times) and the concentration of encapsulated Nile Red was determine by fluorescence spectroscopy. For fluorescence measurements, 20 μL of the purified sample was mixed with 80 μL acetone to a total volume of 100 μL. Fluorescence emission spectra of each sample were collected in triplicate by mixing a 20 μL aliquot of the purified sample with 80 μL of acetone and recording the emission spectra (Nile Red: exc. 535 nm, DPH: exc. 350 nm) of the sample in a micro plate reader. A standard curve for [dye] versus maximal fluorescence intensity was used to calculate the [dye] present in each sample.

### Synthesis of Cy3-Labeled HE₁₂-DNA Micelles:

DNA synthesis was performed on a 1 μmole scale, starting from a universal 1000 Å LCAA-CPG solid support. Coupling efficiency was monitored after removal of the dimethoxytrityl (DMT) 5′ OH protecting groups. DMT-dodecane-diol (cat.# CLP-1114) was purchased from Chemgenes. Cyanine 3 Phosphoramidite (cat.#10-5913-02) was purchased from Glen Research. DMT-dodecane-diol and Cy3 phosphoramidite were dissolved in the appropriate solvent under a nitrogen atmosphere in a glove box (<0.04 ppm oxygen and <0.5 ppm trace moisture). For DMT-dodecane-diol (0.1M, anhydrous acetonitrile) and Cy3 (0.1M, anhydrous acetonitrile) amidites extended coupling times of 5 minutes were used respectively using 0.25M 5-(ethylthio)tetrazole in anhydrous acetonitrile. The Cy3 addition occurred at the final step, yielding the conjugated dye at the 5′ end of the newly synthesized chain.

### Cytotoxicity of BKM120-Loaded Micelles:

### MTS Assay:

The cytotoxicity of micelles was assessed using the CellTiter96 kit from Promega according to the manufacturer's instructions. Briefly, HeLa cells (human cervical cancer) were seeded at a density of 10000 cells per well in a 96-well plate in DMEM media (Invitrogen) supplemented with 10% FBS and AB/AM. BKM120-loaded micelle stock solutions were initially diluted in growth medium to yield final drug concentrations ranging from 0.16 to 70 nM. Cells were incubated for 24, 48, and 72 h in 5% CO₂ at 37° C. After the incubation period, the MTS reagent was added to each well and further incubated for 2 h in 5% CO₂ at 37° C. Subsequently, 96-well plates were allowed to equilibrate at room temperature and the absorbance was read at 490 nm using a BioTek Epoch micro-plate reader. All quantifications were done using GraphPad Prism 5 software.

Potency and cytotoxicity of the BKM120-loaded micelles were measured by in vitro cell viability assay (MTS assay). The study shows an IC₅₀ of BKM120-loaded micelles comparable or better than BKM120 in its naked form.

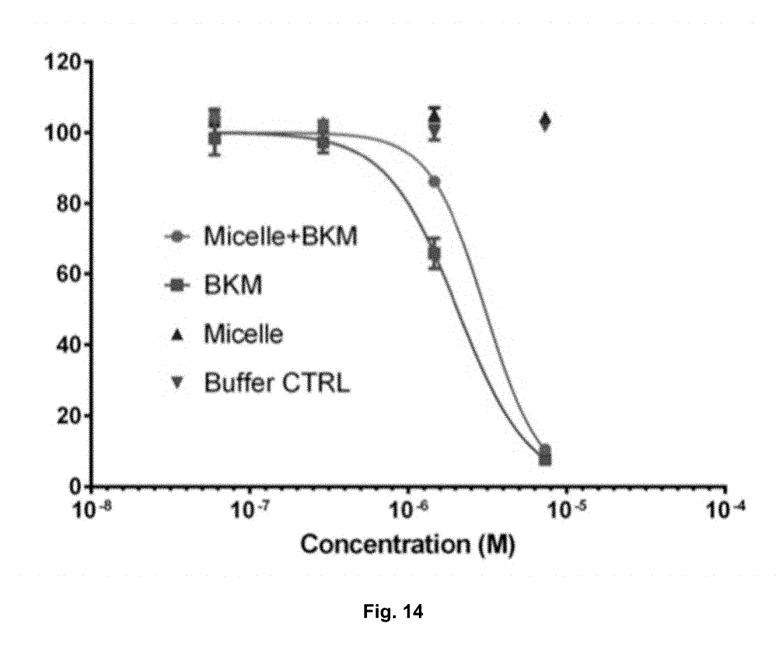

FIG. 14. represents the MTS assay to measure the cytotoxity of BKM120-loaded HE₁₂-DNA micelles. The inhibitory concentration for BKM120-loaded micelles and BKM120 control were determined as 3 μM and 2 μM, respectively. The experiment was performed in quadruplicate

### Cell Studies on Primary B-CLL Lymphocytes:

Apoptosis studies on primary patient CLL cells reveal the high efficiency and potency of BKM120-loaded micelles at inducing apoptosis compared to the drug control.

Primary B-CLL lymphocytes were maintained in RPMI complemented with 10% fetal bovine serum (FBS). BMS2 stromal cell line were maintained in Dulbecco's modified eagle medium (DMEM) supplemented with 10% FBS.BMS2 cells were plate at 70×10⁴ cells/ml in 24 well plates before coculture with primary B-CLL lymphocytes and incubate at 37° C., 5% C02.

For apoptosis assays, 3×10⁶ B-CLL lymphocytes were plated in the presence or absence of stromal cell (BMS2) and incubate 1 h at 37° C., 5% CO₂. Cells were then treated with vehicle, micelle, BKM120, and micelle encapsulated BKM120 for 24 and 48 h.

### AnnexinV/Propidium Iodide Analysis:

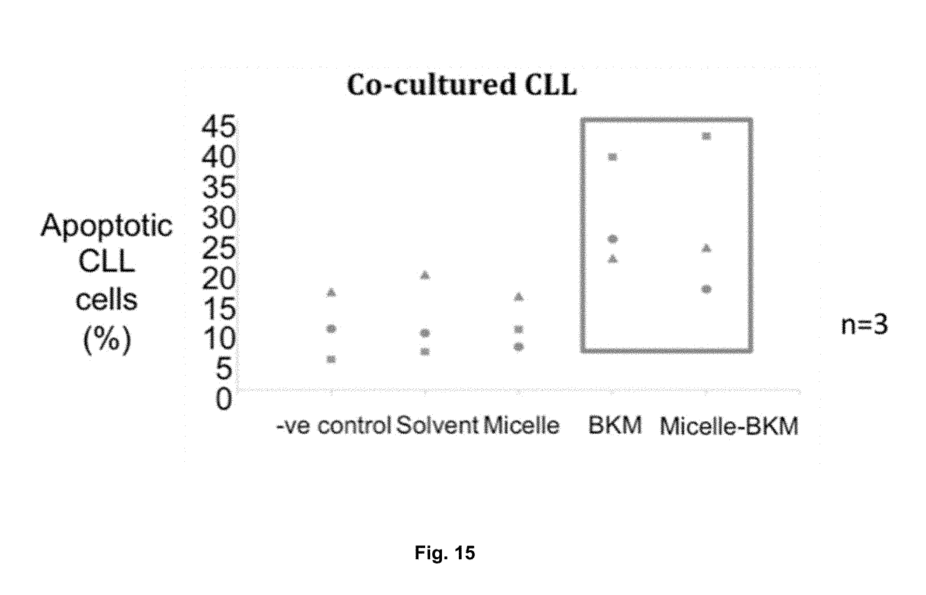

Cells were harvested, washed with PBS then incubated with 1 μl Annexin V APC conjugated plus 0.5 μg/ml propidium iodide in 100 μl binding buffer for 15 min at room temperature. Cells were then analyzed with a FACSCalibur flow cytometer. (See FIG. 15). The BKM-micelle and BKM itself had a similar effect in patient CLL cells.

### Caspase-3 Assay:

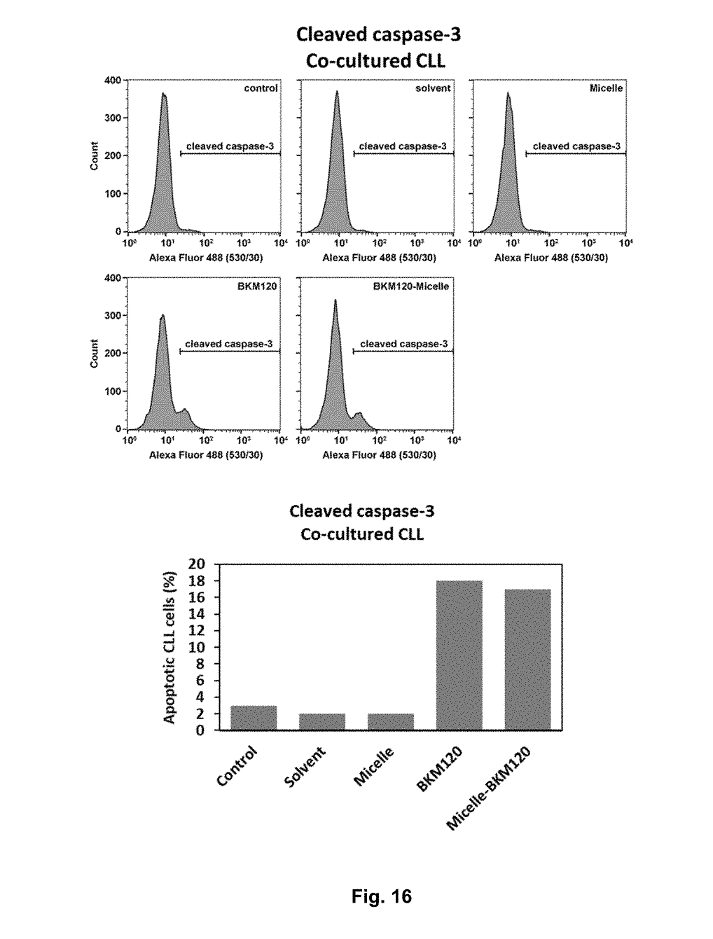

Cells were harvested, washed with PBS then fixed 10 min with 1% paraformaldehyde. After washing, cells were permeabilized and nonspecific sites blocked in PBS containing 3% FBS and 0.01% triton X100. Cells were then incubated 1 h with an anti-caspase-3 FITC conjugated antibody then analyzed with a FACSCalibur flow cytometer. (See FIG. 16). It was found that the same amount caspase was found in both the BKN-micelle and BKM itself.

### General

5-(ethylthio)triazole (ETT) (0.25M in acetonitrile) was purchased from Novabiochem. 3-((bis(diisopropylamino)phosphanyl)oxy)propanenitrile and N,N-Diisopropylamino cyanoethyl phosphonamidic-Cl were purchased from ChemGenes Incorporation. 4,4′ (chloro(phenyl)methylene)bis(methoxybenzene), diisopropylethylamine and triethylamine were purchased from Sigma-Aldrich. 2,2,3,3,4,4,5,5,6,6,7,7,8,8,9,9-hexadecafluorodecane-1,10-diol was purchased from Matrix Scientific.

### Synthesis of DMT-Diol Phosphoramidites (General Protocol)

In an oven-dried round bottom flask, a monoprotected alcohol (e.g. 0.030 mmol) is dissolved in a dry mixture of acetonitrile/tetrahydrofurane 90:10 (300 μl) under an inert atmosphere. Diisopropylethylamine (5.2 μl, 0.030 mmol, 1 eq.) and N,N-Diisopropylamino cyanoethyl phosphonamidic-Cl (6.0 μl, 0.045 mmol, 0.9 eq.) are added. The reaction is allowed to stir at room temperature under an inert atmosphere during 45 minutes. The same reaction can be performed in dry dichloromethane as the solvent if required for some monoprotected alcohol.

5′ Modification of DNA Oligonucleotides with DMT-Diol Phosphoramidites

All coupling reactions were performed with a standard DNA strand made of 19 oligonucleotides. Here

(SEQ ID NO: 1)
′AT′ = TTTTTCAGTTGACCATATA.

The following monoprotected diols were subjected to the coupling to ‘AT’:

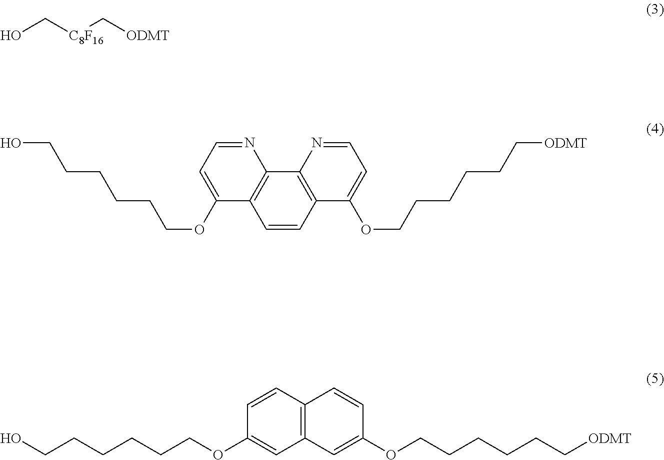

After the automated synthesis of the AT sequence, CPG column is removed from the synthesizer. Under a nitrogen atmosphere, coupling was done using the ‘syringe’ technique: the crude mixture solution (200 μl, 0.1 M) is mixed with an activator solution (200 μl, 0.25 M 5-(ethylthio)tetrazole) in the column using a 1 ml syringe. After ten minutes, the solution is removed from the columns and the strands underwent capping, oxidation and deblocking steps in the synthesizer.

Completed syntheses are deprotected in a 50:50 mixture 28% aqueous ammonium hydroxide solution/methylamine (40 wt. % in water) for 3 hours at 60° C. The crude solution is separated from the solid support and concentrated under reduced pressure at 60° C. This crude solid is re-suspended in 1 mL Millipore water. Sephadex G-25 column and 0.22 μm centrifugal filter are then performed prior to HPLC purification. The resulting solution is quantified by absorbance at 260 nm.

### Attaching Nucleotides at the 5′ End of Modified DNA

Before the deprotection step, five thymidine nucleotides are added on the 5′ end modified DNA by the means of classical automated synthesis. Deprotection step is the same as described before.

HPLC and gel electrophoresis analysis have been performed to measure the yield of the coupling reaction and to characterize the newly synthesized products. The structure of the modified DNA strands has been successfully confirmed by LC-MS.

### HPLC Analysis of 5′ DMT-Diol Modified DNA

Solvents (0.22 μm filtered): 50 mM Triethylammonium acetate (TEAA) buffer (pH 7.5) and HPLC grade acetonitrile. Elution gradient: 3-50% acetonitrile over 30 minutes at 60° C. Column: Hamilton PRP-C18 5 μm 100 Å 2.1×150 mm. For each separation approximately 0.5 OD₂₆₀ of crude strand is injected as a 30 μL solution in Millipore water. Detection is carried out using a diode-array detector, monitoring absorbance at 260 nm.

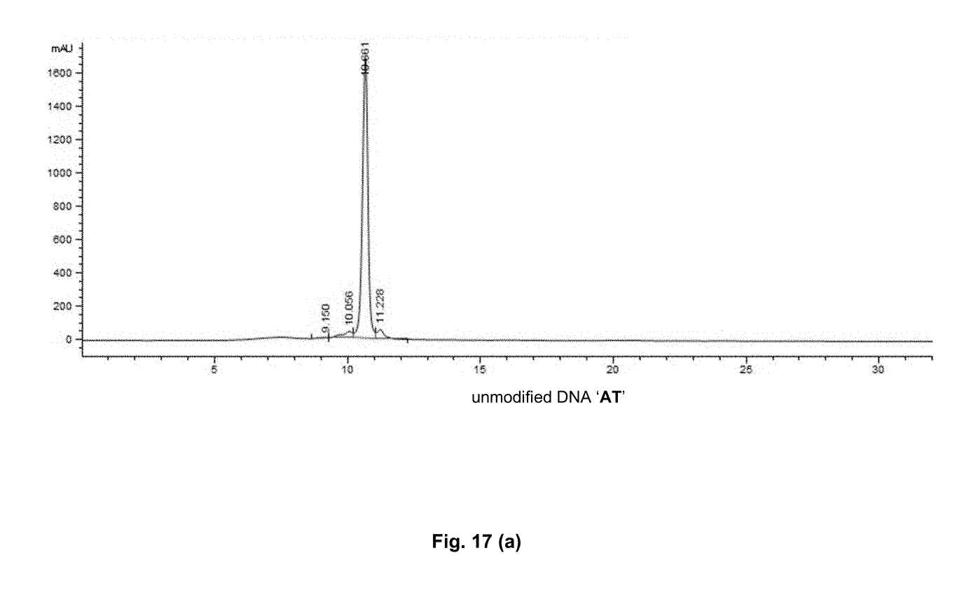

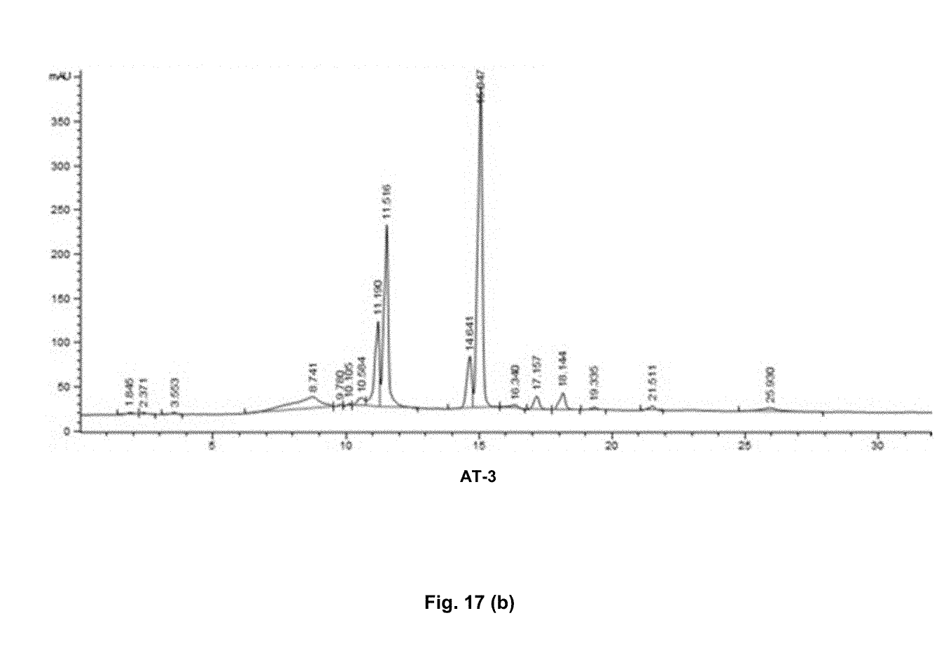

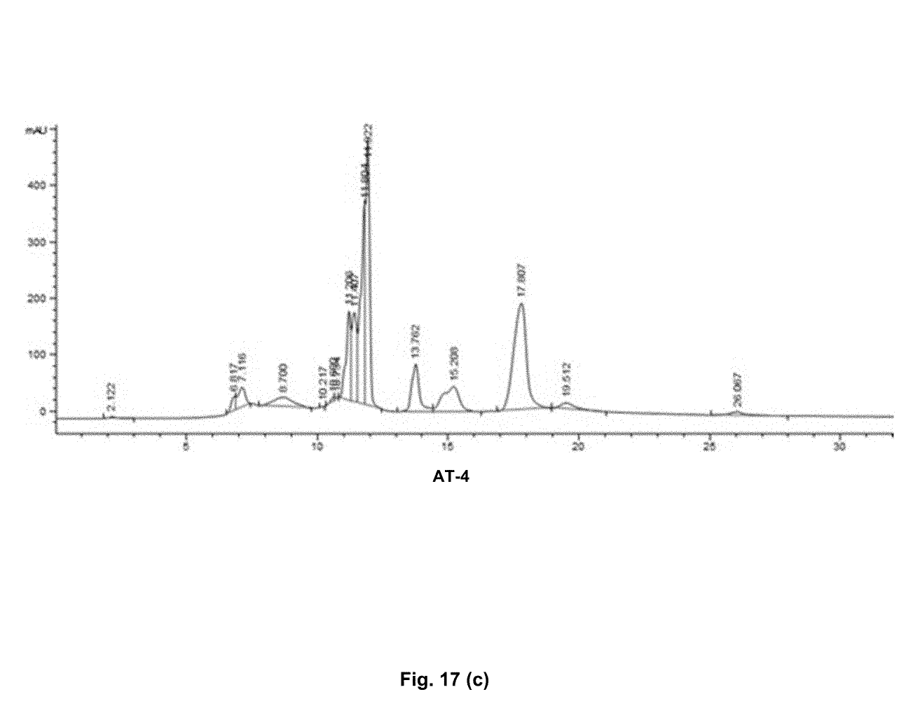

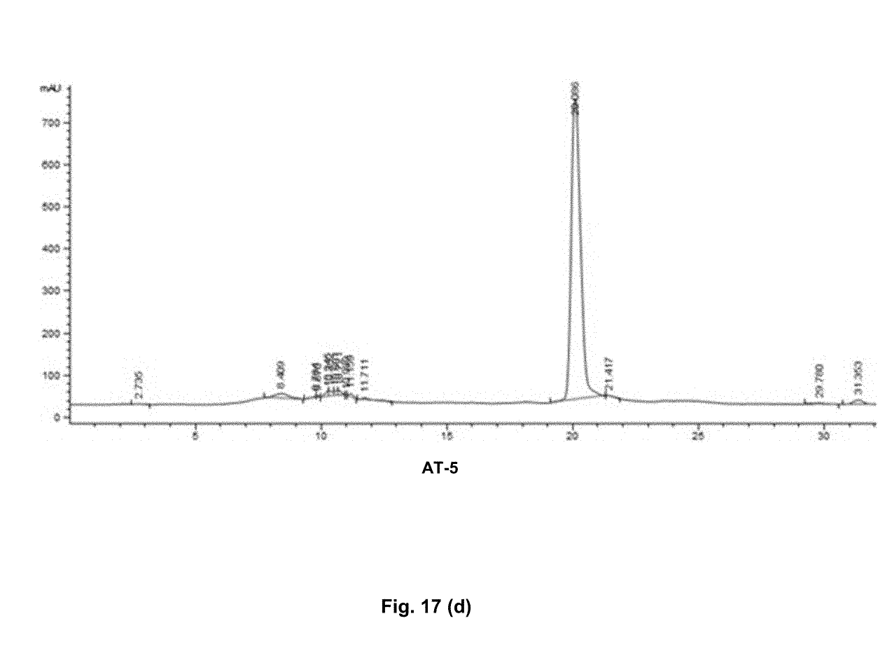

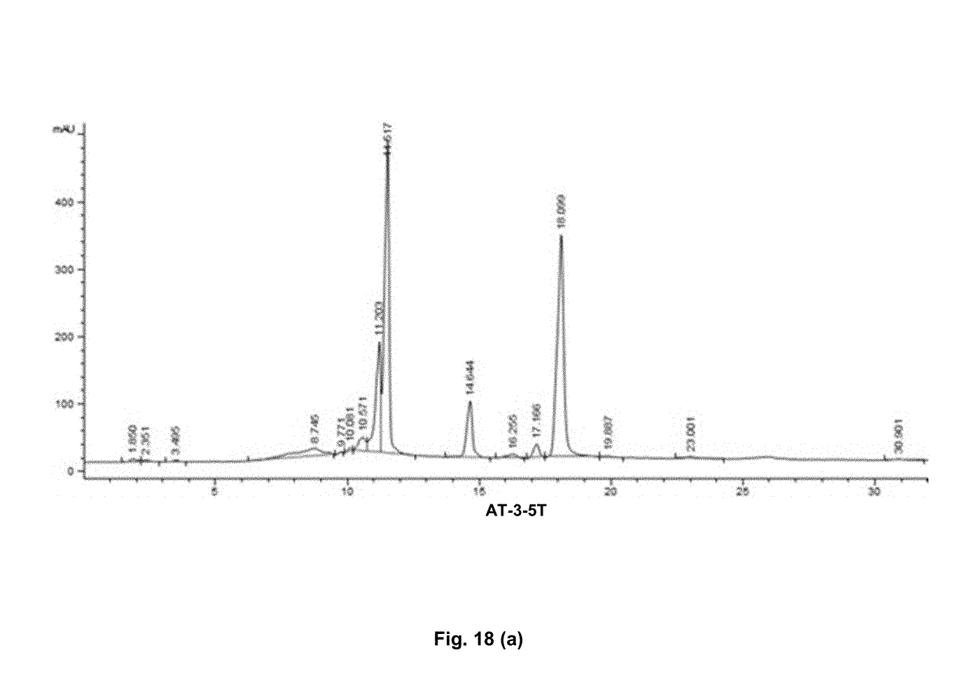

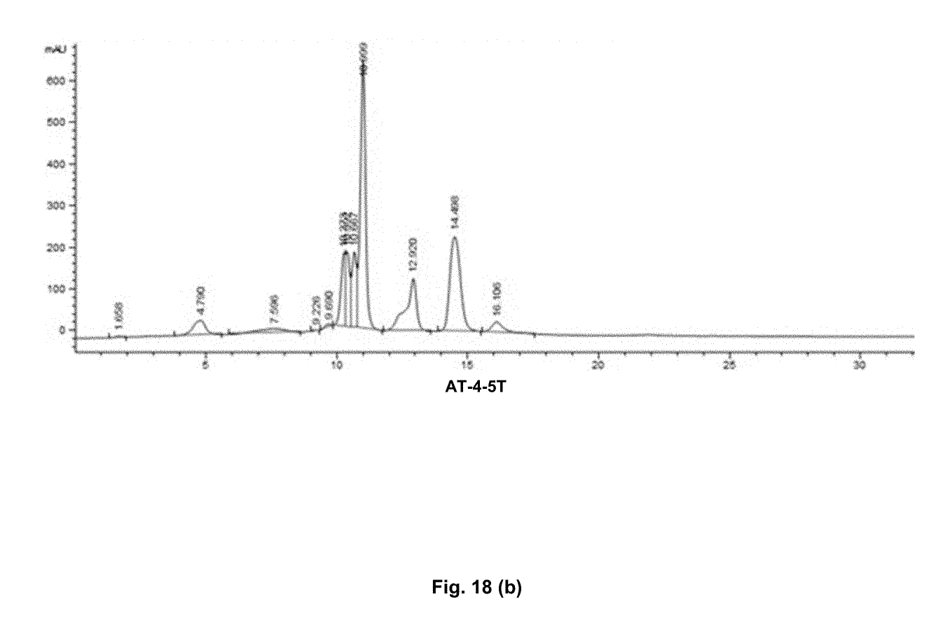

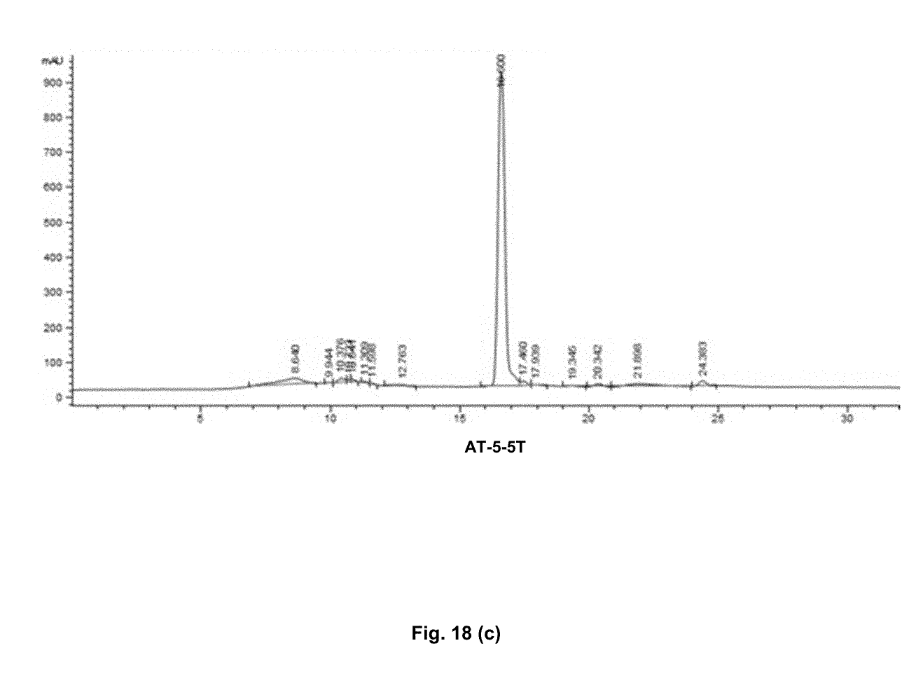

Crude HPLC traces are provided in FIGS. 17 and 18, (in which “AT-3” for example refers to the diol 3 described above bound to sequence ‘AT’ also described above) detection at A=260 nm: the peaks with the highest retention times always are the expected products. The peaks associated to retention times between 10 to 13 minutes were proven to be unmodified DNA strands, DNA strand with an additional phosphate group at the 5′ end or AT sequence dimers.

### PAGE Analysis of DMT-Diol Modified DNA

Denaturing Polyacrylamide Gel Electrophoresis (PAGE) is carried out at room temperature for 30 minutes at 250V followed by 1 hour at 500V. TBE buffer is used and the concentration of urea in the gel was 4M. For each lane 0.01 nmol of modified DNA is loaded as a 4M urea aqueous solution. Sample loading is 0.01 nmol. The DNA bands for all gels were visualized by incubation with GelRed™.

The gel electrophoresis showed that conjugates AT-3, AT-4, AT-5, AT-3-5T, AT-4-5T and AT-5-5T indicated that the expected products were produced.

### Synthesis of Molecule 3

In a 50 ml dry double neck round bottom flask, 2,2,3,3,4,4,5,5,6,6,7,7,8,8,9,9-hexadecafluorodecane-1,10-diol (300 mg, 0.65 mmol, 1 eq.) is dissolved in 10 ml of dry dichloromethane. Triethylamine (270 μl, 1.95 mmol, 3 eq.) is added under stirring. In a 20 ml drop funnel, 4,4′-(chloro(phenyl)methylene)bis(methoxybenzene) is dissolved in 10 ml of dry dichloromethane and is added dropwise on the reaction medium, under vigorous stirring during 15 minutes. The reaction is let under stirring during 24 hours. Solvent is evaporated under reduced pressure. Molecule 3 is purified by column chromatography using a CombiFlash Rf system from Teledyne Isco and a 12 g Gold silica column. A gradient of two solvents, Hexanes/TEA (100:2.5) and DCM (100:2.5), was set up and allowed to get 137 mg of a light yellow oil. Yield: 28%. Purity (calculated through NMR): 91%.

TLC (Hexanes/DCM/TEA: 60:40:2.5): Rf=0.15.

¹H NMR (300 MHz, (CD₃)₂CO): δ (ppm)=7.48 (d, J=6 Hz 2H), 7.28-7.39 (m, 7H), 6.94 (d, J=9 Hz, 4H), 4.15 (t, J=12 Hz, 2H), 3.81 (s, 6H), 3.72 (t, J=27 Hz, 2H).

### Solid-Phase Synthesis of Sequence-Controlled Polymers

Synthesis was performed on a 1 μmole scale, starting from a universal 1000 Å LCAA-CPG solid-support on the DNA synthesizer. Coupling efficiency was monitored after removal of the dimethoxytrityl (DMT) 5′-OH protecting groups. DMT-dodecane-diol (cat.# CLP-1114) and DMT-hexaethyloxy Glycol (cat.# CLP-9765) phosphoramidites were purchased from Chemgenes. DMT-hexaethyloxy Glycol and DMT-dodecane-diol amidites were dissolved (0.1M, anhydrous acetonitrile) under a nitrogen atmosphere in a glove box (<0.04 ppm oxygen and <0.5 ppm trace moisture). Coupling times of 5 minutes were employed with 5-(Ethylthio)tetrazole (0.25M, anhydrous acetonitrile) as the activator. Removal of the DMT protecting group was carried out using 3% dichloroacetic acid in dichloromethane. Importantly, the final DMT group was left attached to the last HE or HEG residue to aid in characterization. Completed syntheses were cleaved from the solid support and the 2-cyanoethyl groups removed by treatment with 28% aqueous ammonium hydroxide solution for 16-18 hours at 65° C. The crude product solution was separated from the solid support and concentrated under reduced pressure at 60° C. This crude solid was re-suspended in 500 μL of 4:1 water:acetonitrile to aid solubility. Table 9 summarizes the sequence-controlled polymers synthesized. Here the 3′ end refers to the end that was attached to the solid support.

TABLE 9

| Molecule | Sequence (5′-xx-3′) |
|---|---|
| HEG₂ | HH |
| HE₁-HEG₂ | DHH |
| HE₂-HEG₂ | DDHH |
| HE₃-HEG₂ | DDDHH |
| HE₆-HEG₂ | DDDDDDHH |

(D = residue obtained from reaction of DMT-
dodecane-diol amidite)
(H = residue obtained from reaction of DMT-
hexaethyloxy Glycol amidite)

All syntheses listed above showed excellent trityl responses for each coupling.

### UV-Vis Analysis of Sequence-Controlled Polymers

The crude product obtained above was analysed by UV-Vis to determine the presence of the DMT end-group to confirm the presence of the full-length product. Furthermore, treatment with DCA was used to release the DMT cation from the 5′-OH of the polymer and provide the sequence-controlled polymer with a free hydroxyl group at each end.

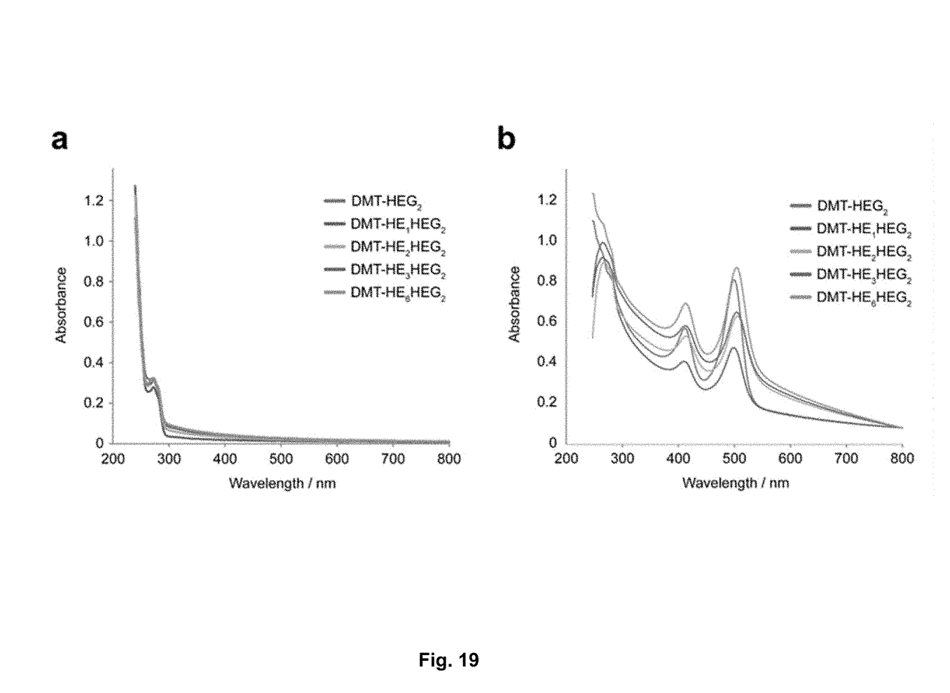

FIG. 19 shows the absorbance study of sequence-controlled polymers in which a) is the absorbance of crude polymer mixtures cleaved from the solid support. The peak at approx. 270 nm corresponds to the DMT group attached to the 5′-OH, thus confirms the presence of the full-length product. In b) The absorbance of the products after addition of DCA to 3% in solution is shown. The cleavage of the DMT-O bond is visible by the peak at ca. 500 nm which corresponds to the DMT+ cation.

### ESI/IT-MS Analysis of Sequence-Controlled Polymers

For Mass spectrometry analysis 5 μL of the crude mixture was diluted by a factor of 80, filtered with a 0.2 μm PTFE filter and used for Electron-Spray Ionization/Ion Trap experiments with a Finnigan LCQ Duo apparatus. A flow rate of 25 μL/min was employed. Results obtained in the negative mode are very consistent with expected results and show almost no byproducts in the crude from the synthesis. In the positive mode, the DMT cation is predominantly visible.

To investigate the removal of the DMT group DCA was added to another 5 μL of the supernatant so as to make a 3% solution of DCA. After five minutes, solvents were evaporated under reduced pressure at 60° C. and resuspended in 40 μL water. After filtration through a 0.2 μm PTFE filter, solvent (8:2 of water:acetonitrile) was added to reach a dilution factor of 80 ready for ESI analysis. Results obtained in the negative and positive modes show the full deprotection of the different molecules with very few byproducts. The relative intensity of the DMT cation peak is much lower than for the first set of analysis showing that resuspension in water provides a simple means to remove most of the DMT from the polymer product. Table 10 summarizes the ESI/IT-MS data for sequence controlled polymers before and after removal of the DMT group from the 5′-OH.

TABLE 10

| | Calculated mass [M − H]⁻ | Mass found [M − H]⁻ |
|---|---|---|
| Polymer | [Da] | [Da] |
| DMT-HEG₂ | 927.41 | 927.49 |
| DMT-HE₁-HEG₂ | 1191.56 | 1191.56 |
| DMT-HE₂-HEG₂ | 1455.71 | 1455.67 |
| DMT-HE₃-HEG₂ | 1719.89 | 1719.76 |
| DMT-HE₆-HEG₂ | 1255.65* | 1256.01* |
| HEG₂ | 625.28 | 625.42 |
| HE₁-HEG₂ | 889.43 | 889.46 |
| HE₂-HEG₂ | 1153.58 | 1153.67 |
| HE₃-HEG₂ | 1417.52 | 1417.67 |
| HE₆-HEG₂ | 1104.59* | 1104.77* |

(*denotes the [M − 2H]² ion. This was used for the longer polymers as the single charged ion [M − H]⁻ is out with the accurate range of the instrument)

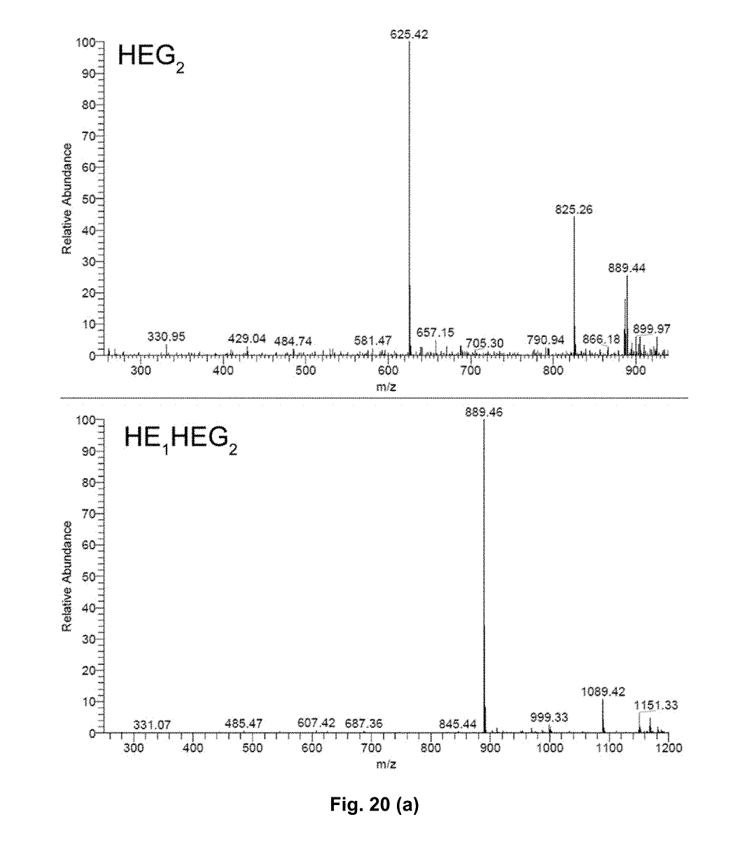

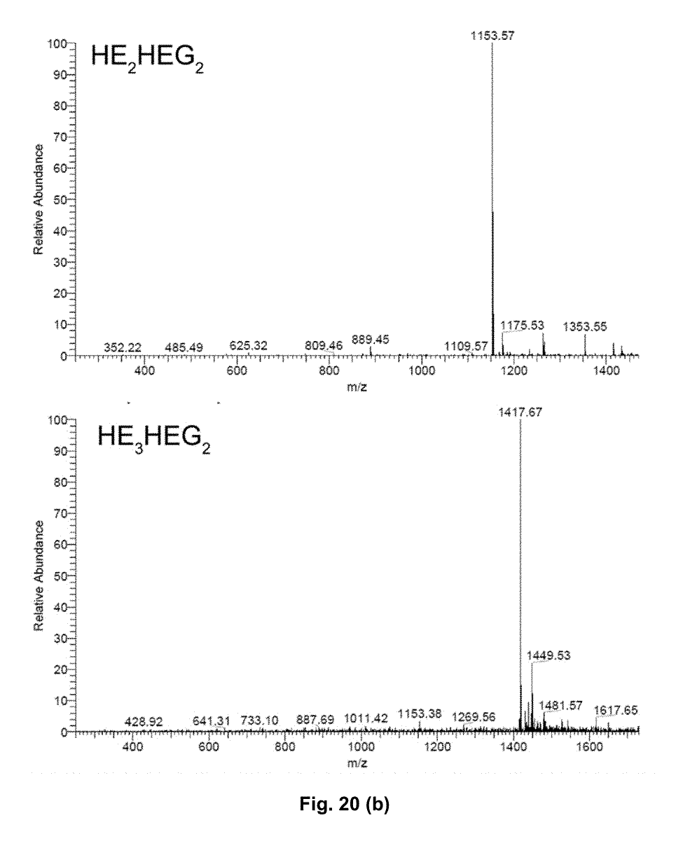

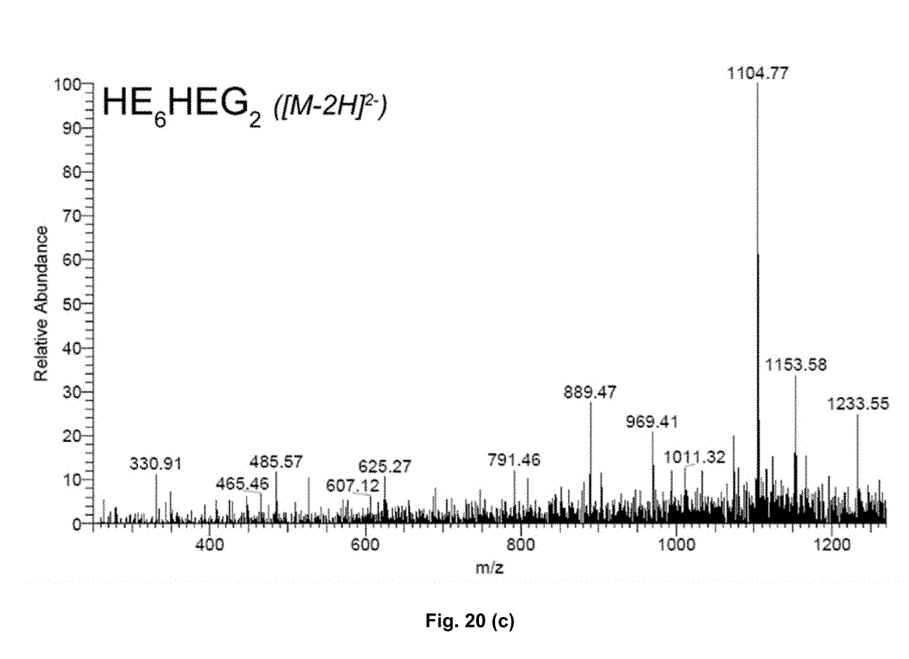

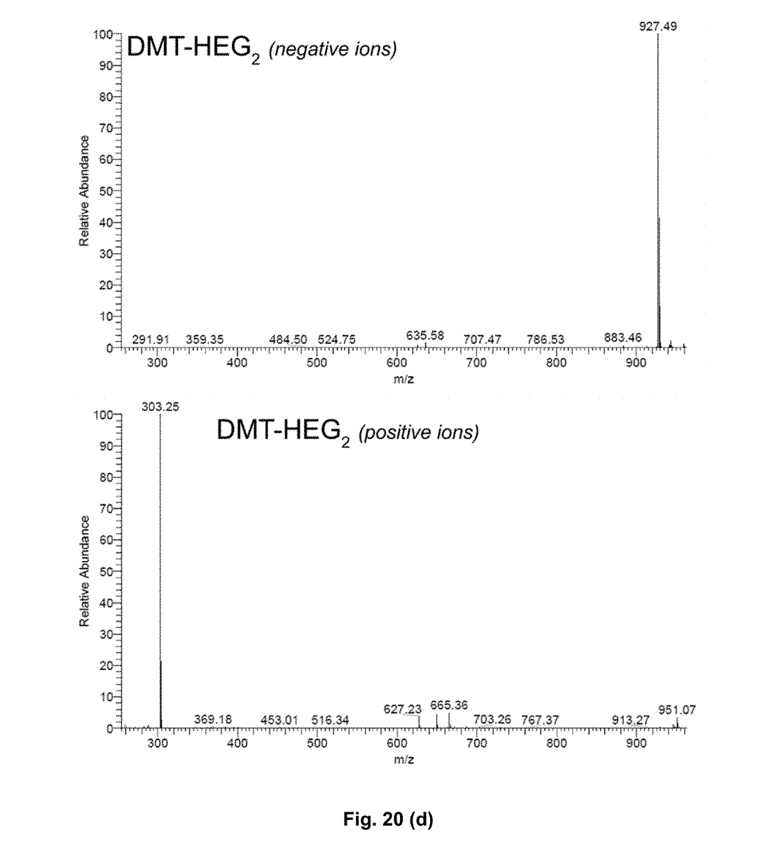

ESI/IT-MS data for sequence-controlled polymers described in table 10 are shown in FIG. 20 (a)-(d).

1. A conjugate of formula (I) 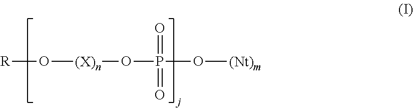
wherein each X is an alkylene chain of 1 to 20 carbon atoms independently selected n times, said alkylene chain carbon atoms being optionally interrupted by one or more of:
i) a cycloalkylene;
ii) an arylene or heteroarylene; and
iii) a heteroatom selected from O or N(R1) wherein R1 is H or a nitrogen substituent, provided that for each of said residue O—X—O, when two or more N or O atoms are present they are separated by two or more carbon atoms of said alkylene chain;
each of said X is optionally independently substituted at any substitutable position by one or more substituent Rx;
Nt is a nucleotide wherein one of a 3′ or 5′ sugar hydroxyl of the 3′ or 5′ end of said Nt is covalently bonded to the phosphorus (P) atom to form the phosphate linking group;
R is H, a suitable hydroxyl protecting group;
n is an integer;
m is an integer; and
j is an integer.

2. The conjugate of claim 1, wherein m is an integer of from 10 to 25; n is an integer of from 3 to 20, j is an integer of from 3 to 20.

3. The conjugate of claim 1, wherein R is H, or a protecting group Fmoc, Trityl, monomethoxy trityl (MMT) or dimethoxy trityl (DMT).

4. The conjugate of claim 1, wherein X is an alkylene chain substituted by Rx which is a F atom.

5. The conjugate of claim 1, wherein X is an alkylene chain interrupted by —O-(arylene or heteroarylene)-O—.

6. The conjugate of claim 1, wherein X is dodecane, hexaethyloxy,

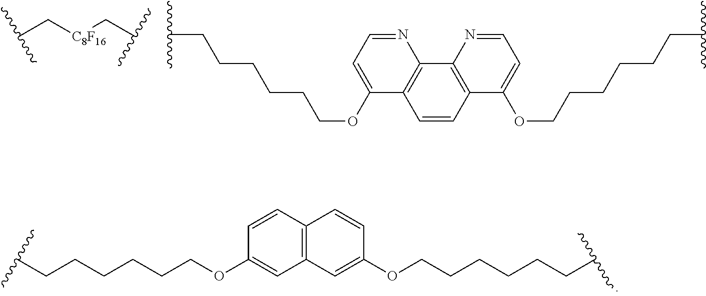

7. The conjugate of claim 1, wherein (Nt)ₘ is a therapeutic nucleotide segment.

8. The conjugate of claim 7, wherein (Nt)ₘ is comprising a silencing mRNA (siRNA), antisense oligonucleotides, micro RNA (miRNA), antagomir (anti-miRNA), aptamer or concatomer.

9. A composition comprising a micelle and one or more compound; wherein said micelle is comprising a conjugate as defined in claim 1, said conjugate having a hydrophilic segment formed by (Nt)ₘ in formula I and a hydrophobic segment sufficient to form a micelle wherein the hydrophobic segment is comprising the residue of —O—(X)n-O— in formula I, said hydrophobic segment forms a hydrophobic inner core and said hydrophilic segment forms the outer shell of said inner core; and wherein said compound is a hydrophobic compound encapsulated in said hydrophobic inner core.

10. A composition of claim 9, wherein said one or more compound is one or more hydrophobic therapeutic agent.

11. The composition of claim 10, wherein in said conjugate, R is H, (X)n is (—CH2CH2-)6, j is an integer of 6 or more and m is an integer of from 10 to 25.

12. The composition of claim 10, wherein in said conjugate, wherein R is H, (X)n is (—CH2CH2-)₆ and j is an integer of 6 or more.

13. The composition of claim 10 wherein said one or more hydrophobic therapeutic agent is comprising anticancer agents, antifungal agents, antibiotics, steroids, anti-inflammatory agents, antiviral agents and vitamins.

14. A method for treating a condition or illness in a subject in need thereof, comprising administering an effective amount of the composition according to claim 9 to a subject in need of treatment.

15. The method of claim 14, wherein said condition or illness is cancer.

16. The method of claim 15 wherein in said conjugate, wherein R is H, (X)n is (—CH2CH2-)₆ and j is an integer of 6 or more.

17. The method of claim 16 wherein said one or more compound is an anticancer agent.


SEQUENCE LISTING (11 sequences)

SEQ ID NO: 1 - DNA, 19 bp, Artificial Sequence
  AT sequence of some conjugates presented in Table 1
tttttcagttgaccatata

SEQ ID NO: 2 - DNA, 14 bp, Artificial Sequence
  Acomp sequence of conjugate presented in Table 1
tatatggtcaactg

SEQ ID NO: 3 - DNA, 19 bp, Artificial Sequence
  Sequence of conjugates presented in Table 4
tttttcagttgaccatata

SEQ ID NO: 4 - DNA, 21 bp, Artificial Sequence
  Sequence of conjugate presented in Table 6
  misc_feature (1)..(21): DNA/RNA hybrid
uuaauuaaagacuucaagcgg

SEQ ID NO: 5 - DNA, 21 bp, Artificial Sequence
  Sequence of conjugate presented in Table 6
  misc_feature (1)..(21): DNA/RNA hybrid
gcuugaagucuuuaauuaatt

SEQ ID NO: 6 - DNA, 26 bp, Artificial Sequence
  Sequence of conjugate presented in Table 6
  misc_feature (1)..(26): DNA/RNA hybrid
gcuugaagucuuuaauuaattaaaaa

SEQ ID NO: 7 - DNA, 21 bp, Artificial Sequence
  Sequence of conjugate presented in Table 6
  misc_feature (1)..(21): DNA/RNA hybrid
gcuugaagucuuuaauuaatt

SEQ ID NO: 8 - DNA, 26 bp, Artificial Sequence
  Sequence of conjugate presented in Table 6
  misc_feature (1)..(26): DNA/RNA hybrid
uuaauuaaagacuucaagcggaaaaa

SEQ ID NO: 9 - RNA, 23 nt, Artificial Sequence
  Sequence of conjugate presented in Table 6
  misc_feature (21)..(21): cm
  misc_feature (22)..(22): n = 2′-O-methyladenine
  misc_feature (23)..(23): n = cm
auugguauucagugugaugannn

SEQ ID NO: 10 - RNA, 21 nt, Artificial Sequence
  Sequence of conjugate presented in Table 6
gucaucacacugaauaccaau

SEQ ID NO: 11 - RNA, 23 nt, Artificial Sequence
  Sequence of conjugate presented in Table 6
  misc_feature (21)..(21): n = cm
  misc_feature (22)..(22): n = 2′-O-methyladenine
  misc_feature (23)..(23): n = cm
auugguauucagugugaugannn